(19) United States
(12) Patent Application Publication
(10) Pub. No.: US 2024/0000354 A1
(43) Pub. Date: Jan. 4, 2024

(54) DRIVING CHARACTERISTIC DETERMINATION DEVICE, DRIVING CHARACTERISTIC DETERMINATION METHOD, AND RECORDING MEDIUM

(71) Applicant: Panasonic Intellectual Property Management Co., Ltd., Osaka (JP)

(72) Inventors: Nobuyuki KUNIEDA, KANAGAWA (JP); Koichi EMURA, KANAGAWA (JP); Akihiro OHMOTO, TOKYO (JP)

(73) Assignee: Panasonic Intellectual Property Management Co., Ltd., Osaka (JP)

(21) Appl. No.: 18/368,650

(22) Filed: Sep. 15, 2023

Related U.S. Application Data
(63) Continuation of application No. PCT/JP2022/005706, filed on Feb. 14, 2022.

(30) Foreign Application Priority Data
Mar. 25, 2021 (JP) 2021-052309

(51) Int. Cl. A61B 5/16; G07C 5/08; A61B 5/18

(52) U.S. Cl. CPC A61B 5/163; G07C 5/0808; A61B 5/18; B60K 35/00

(57) ABSTRACT

A driving characteristic determination device includes a hardware processor connected to a memory. The processor detects driving information indicating at least one of driving behavior for a vehicle by a driver, biological information of the driver during driving, and behavior of the vehicle. The processor calculates, on the basis of the driving information, numerical values indicating whether a cognitive function of the driver is high or low. The processor analyzes the numerical values indicating whether the cognitive function is high or low. The numerical values are analyzed as cognitive function characteristics relative to one or more different brain functions. The processor outputs information about an analysis result obtained by the analysis of the numerical values.

### CROSS-REFERENCE TO RELATED APPLICATIONS

This application is a continuation of International Application No. PCT/JP2022/005706, filed on Feb. 14, 2022, which claims the benefit of priority of the prior Japanese Patent Application No. 2021-052309, filed on Mar. 25, 2021, the entire contents of which are incorporated herein by reference.

### FIELD

Embodiments described herein relate generally to a driving characteristic determination device, a driving characteristic determination method, and a recording medium.

### BACKGROUND

In analysis of traffic accidents due to human factors, “delay of discovery” accounts for about 80% of the human factors such as front carelessness (including cognitive distracted driving and visual-manual distracted driving) and failure in safety confirmation (see Institute for Traffic Accident Research and Data Analysis: “Traffic accident statistical table data: classified by human factors and accident types, total number of accidents (first party)—vehicle”, 2020). These statistics indicate that the main factor is cognition part from whole driving process “cognition, decision, action”. As a factor that influences decline of a cognitive function related to driving, exemplified are drowsiness, alcohol and drugs, aging, dementia, and psycho-neurologic disease including higher cortical dysfunction (see Masaru Mimura, Yoshio Fujita “Automobile Driving and Cognitive Function About the Driving”, Japanese Journal of Geriatrics, vol. No. 2, pp. 191-196, 2018). Thus, it can be considered that traffic accidents can be reduced if it is possible to prevent decline of the recognition function during driving caused by various factors. A cognitive function of a person, a cognitive function of a driver, behavior analysis for a driver during driving, and so forth have been researched from various viewpoints as described in the following list of literature: “Supervised by Takao Suzuki, “Basics of mild cognitive impairment (MCI)—aiming at effective prevention of dementia”, p. 225, Igaku-Shoin Ltd., 2015”; “JAPANESE SOCIETY OF NEUROLOGY: “Dementia medical care guideline 2017”, Igaku-Shoin Ltd., pp. 19-22, 2017”; “Shinya Iida, Noriaki Kato, Kenji Hachisuka, Satoru Saeki, “Determination of driving ability of elderly people”, Japanese Journal of Geriatrics, vol. 55, No. 2, pp. 202-207, 2018”; “Naoto Kamimura: “Driving Ability and Assessment of Fitness to Drive for Dementia/Cognitive Decline”, The Journal of the International Association of Traffic and Safety Sciences, vol. 42, No. 3, pp. 12-22, 2018”; “Katsuya Urakami: “Dementia and driving”, Society of Automotive Engineers of Japan, Inc., vol. 71, No. 12, pp. 90-95, 2017”; “Ryoko Fukuda, Fumio Harada, Taisaku Okumura, “Vehicle for a Super-Aged Society: Focusing on One's Way of Being”, Cognitive Studies, 25(3), pp. 259-278, 2018. 09”; “Shinya Takagi, Keiichi Yamada, “On the Relationship between Behavior of a Vehicle and Reaction Time of the Driver”, Transactions of the Society of Automotive Engineers of Japan, Vol. 43, No. 5, pp. 1131-1137, 2012”; “Li Bo, Zhang Xiaolin, Makoto Sato, “Pitch Angle Estimation Using a Vehicle Mounted Monocular Camera for Vehicle Target Range Measurement”, The journal of the Institute of Image Information and Television Engineers, vol. 69, No. 4, pp. J169-J176, 2015”; “Tatsuki Kamisaka, Masafumi Noda, Yoshito Mekada, Daisuke Deguchi, Ichiro Ide, Hiroshi Murase, “Prediction of driving behavior using driver's gaze information”, IEICE technical report, MI, medical image 111 (49), 105-110, 2011-05-12”; “Akito Yamasaki, Pongsathorn Raksincharoensak, Motoki Shino, “Extraction of Driver's Gaze Region by Face Direction Estimation Using On-Board Cameras”, Transactions of the Society of Automotive Engineers of Japan, Vol. 48, No. 5, pp. 1113-1119, 2017”; “Masanari Takagi, Hironobu Fujiyoshi, “Traffic Sign Recognition Using SIFT features”, IEEJ Transactions on Electronics, Information and Systems, vol. 129, No. 5, pp. 824-831, 2009”; “David W. Eby, Lisa J. Molnar, Paula S. Kartje (Edited and translated by Etsuo Horikawa, Tomoko Mine), “Maintaining safe mobility in an aging society”, Kyoto University Press, pp. 15-33, 2020”, “Tsuneo Matsuura, “Safety psychology of elderly driver”, University of Tokyo Press, pp. 48-62, 2017”; and “Kazuyoshi Isaji, Naohiko Tsuru, Takahiro Wada, Shun'ichi Doi, Hiroshi Kaneko, “Analysis of the Brake Initiation Timing On the basis of Performance Index for Approach and Alienation”, Transactions of the Society of Automotive Engineers of Japan, vol. 41, No. 3, pp. 593-598, 2010”.

Japanese Patent Application Laid-open No. 2009-101714 discloses a driving and driving assist device that detects a state where driving ability has declined due to drinking, falling asleep, etc., and notifies a driver of decline of driving ability. Japanese Patent Application Laid-open No. 2019-124975 discloses a determination system for risk of dementia that detects a traffic offense that tends to be committed when a cognitive function has declined, and determines whether a driver is enabled to drive.

However, in Japanese Patent Application Laid-open No. 2009-101714, decline of the driving ability is estimated by detecting an arousal level or a drunk state, and decline of the cognitive function is not estimated on the basis of a cognitive function mechanism of a brain. In Japanese Patent Application Laid-open No. 2019-124975, decline of the cognitive function that does not cause a traffic offense is not evaluated because decline of the cognitive function cannot be determined unless a traffic offense is actually caused. Additionally, there is no mention of assisting driving behavior corresponding to a decline of the cognitive function.

### SUMMARY

A driving characteristic determination device according to the present disclosure includes a hardware processor connected to a memory. The hardware processor is configured to detect driving information indicating at least one of driving behavior for a vehicle by a driver, biological information of the driver during driving, and behavior of the vehicle. The hardware processor is configured to calculates, on the basis of the driving information, numerical values indicating whether a cognitive function of the driver is high or low. The hardware processor is configured to analyze the numerical values indicating whether the cognitive function is high or low. The numerical values are analyzed as cognitive function characteristics relative to one or more different brain functions. The hardware processor is configured to outputs information about an analysis result obtained by the analysis of the numerical values.

### BRIEF DESCRIPTION OF THE DRAWINGS

FIG. 1 is a diagram for explaining a state of decline of a cognitive function characteristic along with aging;

FIG. 2 is a diagram for explaining a cognitive function characteristic determined by a driving characteristic determination device according to an embodiment;

FIG. 3 is a block diagram illustrating an example of a schematic configuration of the driving characteristic determination device according to the embodiment;

FIG. 4 is an external view illustrating an example of a cockpit of a vehicle on which the driving characteristic determination device according to the embodiment is mounted;

FIG. 5 is a functional block diagram illustrating an example of a functional configuration of the driving characteristic determination device according to the embodiment;

FIG. 6 is a diagram for explaining an example of information to be detected by a driving state detection unit;

FIG. 7 is a flowchart illustrating an example of a processing procedure of calculating an evaluation score of a cognitive function by a cognitive function calculation unit;

FIG. 8 is a diagram for explaining a relation between cognitive function characteristics relative to different brain functions and driving behavior that is caused during driving;

FIG. 9 is a first diagram for explaining an example of content of assist performed by the driving characteristic determination device in accordance with the cognitive function characteristic;

FIG. 10 is a second diagram for explaining an example of content of assist performed by the driving characteristic determination device in accordance with the cognitive function characteristic;

FIG. 11 is a first diagram for explaining a specific method for selecting a function of assisting a driver when the cognitive function characteristic declines;

FIG. 12 is a second diagram for explaining a specific method for selecting a function of assisting the driver when the cognitive function characteristic declines;

FIG. 13 is a first diagram illustrating an example of information presented to a vehicle in a case where the driving characteristic determination device activates a training mode;

FIG. 14 is a second diagram illustrating an example of information presented to the vehicle in a case where the driving characteristic determination device activates the training mode;

FIG. 15 is a first diagram illustrating an example of information presented to the vehicle in a case where the driving characteristic determination device activates a driving assist mode;

FIG. 16 is a second diagram illustrating an example of information presented to the vehicle in a case where the driving characteristic determination device activates the driving assist mode;

FIG. 17 is a diagram illustrating an example of information presented to the vehicle in a case where the driving characteristic determination device activates the training mode and the driving assist mode at the same time;

FIG. 18 is a first diagram illustrating an example of an operating state of the training mode;

FIG. 19 is a second diagram illustrating an example of an operating state of the training mode;

FIG. 20 is a flowchart illustrating an example of a processing procedure performed by the driving characteristic determination device;

FIG. 21 is a diagram for explaining an effect of a modification of the embodiment; and

FIG. 22 is a diagram for explaining another method for calculating the cognitive function characteristic.

### DETAILED DESCRIPTION

The following describes an embodiment of a driving characteristic determination device according to the present disclosure with reference to the drawings.

Description of Cognitive Function Characteristic

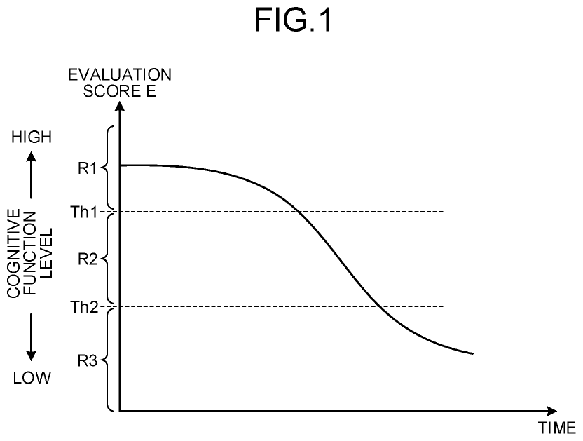

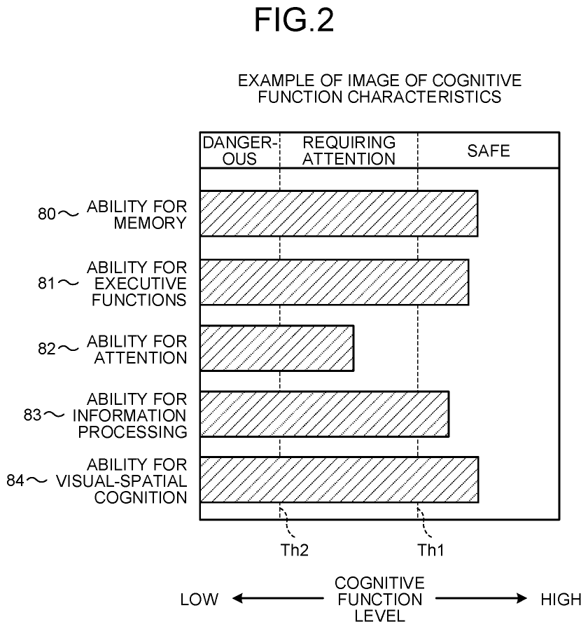

The following describes a cognitive function characteristic of a driver with reference to FIG. 1 and FIG. 2. FIG. 1 is a diagram for explaining a state of decline of the cognitive function characteristic along with aging. FIG. 2 is a diagram for explaining the cognitive function characteristic determined by the driving characteristic determination device according to the embodiment.

As illustrated in FIG. 1, the cognitive function characteristic may decline over time. A numerical value indicating whether a cognitive function is high or low is referred to as an evaluation score E of the cognitive function. In a case where the evaluation score E of the cognitive function calculated by using an appropriate evaluation method is larger than a first threshold Th1, that is, in a case where the evaluation score E of the cognitive function falls within a region R1, determination is made such that the cognitive function is in a state capable of keeping safe driving. In a case where the evaluation score E of the cognitive function is smaller than the first threshold Th1 and larger than a second threshold Th2 that is smaller than the first threshold Th1, that is, in a case where the evaluation score E of the cognitive function falls within a region R2, determination is made such that the cognitive function is in a state of “requiring attention” in which safe driving is hindered from being continued. Moreover, in a case where the evaluation score E of the cognitive function is smaller than the second threshold Th2, that is, the evaluation score E of the cognitive function falls within a region R3, determination is made such that the cognitive function is in a “dangerous” state where a cognitive function level has declined to a level at which driving is difficult to be continued.

Additionally, in a case where the driver performs cognitive distracted driving or visual-manual distracted driving, or in a case where ability for attention has temporarily declined, the cognitive function declines as illustrated in FIG. 1. Also in a case where the cognitive function has declined due to aging or Mild Cognitive Impairment (MCI), the same cognitive function as that illustrated in FIG. 1 can be evaluated, and variation thereof is observed.

A driving characteristic determination device 10 according to the present embodiment converts the cognitive function of the driver into a numerical value. The driving characteristic determination device 10 then analyzes a state of the cognitive function characteristic on the basis of the numerical value. Moreover, the driving characteristic determination device 10 performs an appropriate driving assist on the basis of a result of the analysis.

The cognitive function can be classified into different cognitive functions respectively relative to different brain parts (brain functions) (see Supervised by Takao Suzuki, “Basics of mild cognitive impairment (MCI)—aiming at effective prevention of dementia”, p. 225, Igaku-Shoin Ltd., 2015). With reference to “Supervised by Takao Suzuki, “Basics of mild cognitive impairment (MCI)—aiming at effective prevention of dementia”, p. 225, Igaku-Shoin Ltd., 2015”, the driving characteristic determination device 10 according to the present embodiment evaluates different cognitive functions illustrated in FIG. 2 as evaluation targets. Specifically, the cognitive functions include ability for memory 80, ability for executive function 81, ability for attention 82, ability for information processing 83, and ability for visual-spatial cognition 84. Influence on driving due to decline of each of the cognitive functions is described in “Masaru Mimura, Yoshio Fujita “Automobile Driving and Cognitive Function About the Driving”, Japanese Journal of Geriatrics, vol. 55, No. 2, pp. 191-196, 2018”, “Shinya lida, Noriaki Kato, Kenji Hachisuka, Satoru Saeki: “Determination of driving ability of elderly people”, Japanese Journal of Geriatrics, vol. 55, No. 2, pp. 202-207, 2018”, “Naoto Kamimura: “Driving Ability and Assessment of Fitness to Drive for Dementia/Cognitive Decline”, The Journal of the International Association of Traffic and Safety Sciences, vol. 42, No. 3, pp. 12-22, 2018”, and “Katsuya Urakami: “Dementia and driving”, Society of Automotive Engineers of Japan, Inc., vol. 71, No. 12, pp. 90-95, 2017”. Five cognitive functions are selected as evaluation targets in FIG. 2, whereas the evaluation target may be only one cognitive function, or may be an optional combination of two or more cognitive functions. Alternatively, a cognitive function that is not described herein may be selected as the evaluation target.

The ability for memory 80 is a cognitive function of storing a new experience and reproducing the experience in consciousness or behavior (see JAPANESE SOCIETY OF NEUROLOGY: “Dementia medical care guideline 2017”, Igaku-Shoin Ltd., pp. 19-22, 2017). In light of driving behavior, for example, the ability for memory is reflected in ability to hold information indicated in a traffic sign (or a road sign), ability to remember where to go, etc. (see Shinya lida, Noriaki Kato, Kenji Hachisuka, Satoru Saeki: “Determination of driving ability of elderly people”, Japanese Journal of Geriatrics, vol. 55, No. 2, pp. 202-207, 2018).

The ability for executive function 81 is a cognitive function of planning and executing a matter with a purpose, and advances the matter while feeding back a result thereof (see JAPANESE SOCIETY OF NEUROLOGY: “Dementia medical care guideline 2017”, Igaku-Shoin Ltd., pp. 19-22, 2017). In light of driving behavior, for example, the ability for executive function 81 is reflected in ability to correctly step on an accelerator and a brake pedal, ability to perform pieces of information processing, etc. (see Shinya lida, Noriaki Kato, Kenji Hachisuka, Satoru Saeki: “Determination of driving ability of elderly people”, Japanese Journal of Geriatrics, vol. 55, No. 2, pp. 202-207, 2018).

The ability for attention 82 is a cognitive function as a basis of receiving and selecting a surrounding stimulus, and performing consistent behavior therefor (see JAPANESE SOCIETY OF NEUROLOGY: “Dementia medical care guideline 2017”, Igaku-Shoin Ltd., pp. 19-22, 2017). In light of driving behavior, for example, the ability for attention 82 is reflected in ability to pay attention to surrounding environment such as a traffic sign or traffic lights (see Shinya lida, Noriaki Kato, Kenji Hachisuka, Satoru Saeki: “Determination of driving ability of elderly people”, Japanese Journal of Geriatrics, vol. 55, No. 2, pp. 202-207, 2018).

The ability for information processing 83 is a cognitive function of executing designated work within a given time (see Supervised by Takao Suzuki, “Basics of mild cognitive impairment (MCI)—aiming at effective prevention of dementia”, p. 225, Igaku-Shoin Ltd., 2015). In light of driving behavior, for example, the ability for information processing 83 is reflected in ability of finding and coping with danger during driving, etc. (see Tsuneo Matsuura: “Safety psychology of elderly driver”, University of Tokyo Press, pp. 48-62, 2017).

The ability for visual-spatial cognition 84 is a cognitive function of processing information seen by eyes to grasp a state of a space. In light of driving behavior, for example, the ability for visual-spatial cognition 84 is reflected in ability to correctly keep a sense of distance to a preceding vehicle, ability to prevent the vehicle from departing from a lane when the vehicle goes round a curve, etc. (see Shinya lida, Noriaki Kato, Kenji Hachisuka, Satoru Saeki: “Determination of driving ability of elderly people”, Japanese Journal of Geriatrics, vol. 55, No. 2, pp. 202-207, 2018).

These cognitive functions are all known to decline as illustrated in FIG. 1. Therefore, as illustrated in FIG. 2, a degree of each of the cognitive functions described above can be evaluated on the basis of a large/small relation with respect to the first threshold Th1 and the second threshold Th2. Note that, in FIG. 2, a horizontal axis is normalized to be illustrated, and the first threshold Th1 and the second threshold Th2 are not necessarily the same for the respective cognitive functions.

Entire Configuration of Driving Characteristic Determination Device

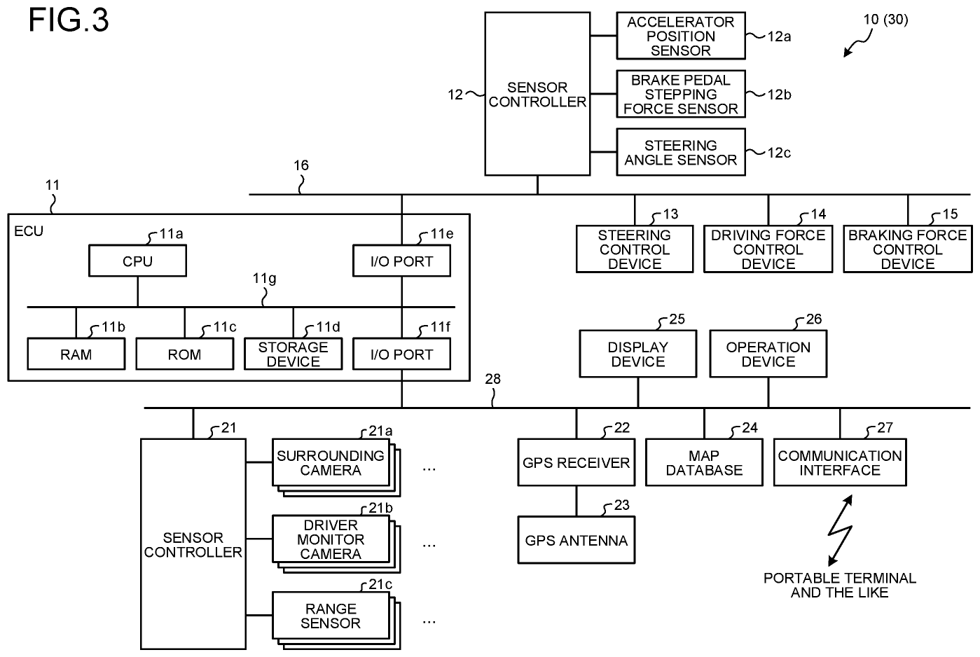

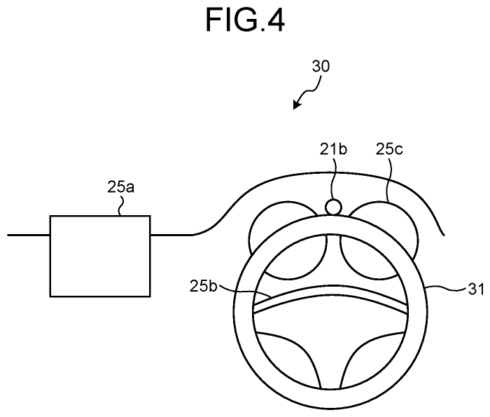

With reference to FIG. 3 and FIG. 4, the following describes the entire configuration of the driving characteristic determination device 10. FIG. 3 is a block diagram illustrating an example of a schematic configuration of the driving characteristic determination device according to the embodiment. FIG. 4 is an external view illustrating an example of a cockpit of a vehicle on which the driving characteristic determination device according to the embodiment is mounted.

The driving characteristic determination device 10 calculates cognitive functions of the driver of a vehicle 30, and performs a driving assist corresponding to a decline of the cognitive functions of the driver.

The driving characteristic determination device 10 includes an electronic control unit (ECU) 11, sensor controllers 12 and 21, a steering control device 13, a driving force control device 14, a braking force control device 15, a GPS receiver 22, a GPS antenna 23, a map database 24, a display device 25, an operation device 26, and a communication interface 27.

The ECU 11 is configured as a computer including a central processing unit (CPU) 11a, a random access memory (RAM) 11b, and a read only memory (ROM) 11c, for example. A storage device 11d constituted of a hard disk drive (HDD) and so forth may be incorporated in the ECU 11. The ECU 11 also includes I/O (Input/Output) ports 11e and 11f for enabling detection signals and various kinds of information to be transmitted/received to/from various sensors. The I/O port 11e is connected to a bus line 16 through which information related to driving control of the vehicle 30 flows, and controls input/output of information related to a control system that performs various kinds of driving assist for the vehicle 30. The I/O port 11f is connected to a bus line 28 through which information related to an information system of the vehicle 30 flows, and controls input/output of information related to detection of driving behavior of the driver, and information presented to the driver.

The RAM 11b, the ROM 11c, the storage device 11d, and the I/O ports 11e and 11f of the ECU 11 are configured to be able to transmit/receive various kinds of information to/from the CPU 11a via an internal bus 11g.

In the ECU 11, various kinds of processing performed by the driving characteristic determination device 10 are controlled by the CPU 11a (an example of the hardware processor) by reading and executing a computer program installed in the ROM 11c.

The computer program executed by the driving characteristic determination device 10 according to the present embodiment may be embedded and provided in the ROM 11c, or may be recorded and provided in a computer-readable recording medium such as a CD-ROM, a flexible disk (FD), a CD-R, and a digital versatile disc (DVD), as an installable or executable file.

Moreover, the computer program executed by the driving characteristic determination device 10 according to the present embodiment may be stored in a computer connected to a network such as the Internet and provided by being downloaded via the network. Additionally, the computer program executed by the driving characteristic determination device 10 according to the present embodiment may be provided or distributed via a network such as the Internet.

The storage device 11d stores a table and so forth used for calculating the evaluation score E of the cognitive function of the driver. Details thereof will be described later.

The sensor controller 12 acquires a sensor output for detecting behavior of the vehicle 30, and passes the sensor output to the ECU 11. To the sensor controller 12, an accelerator position sensor 12a, a brake pedal stepping force sensor 12b, a steering angle sensor 12c, and so forth are connected. The sensors connected to the sensor controller 12 are not limited to these examples, but another sensor may be connected thereto.

The accelerator position sensor 12a detects a pressing degree (or opening degree) of an accelerator of the vehicle 30.

The brake pedal stepping force sensor 12b detects stepping force applied to a brake pedal of the vehicle 30, namely, pressing force for the brake pedal.

The steering angle sensor 12c detects a steering direction and a steering amount of a steering wheel of the vehicle 30.

To the bus line 16, the steering control device 13, the driving force control device 14, and the braking force control device 15 are connected. These devices form what is called an Advanced Driver Assistance System (ADAS) system in which the devices cooperate with each other to control behavior of the vehicle 30 on the basis of various kinds of sensor information acquired by the sensor controller 12 and various kinds of sensor information acquired by the sensor controller 21.

The steering control device 13 controls a steering angle of the vehicle 30 in accordance with an instruction from the ECU 11.

The driving force control device 14 controls driving force of the vehicle 30 in accordance with an instruction from the ECU 11. Specifically, the driving force control device 14 controls the opening degree of the accelerator of an engine of the vehicle 30 in accordance with an instruction from the ECU 11.

The braking force control device 15 controls braking force of the vehicle 30 in accordance with an instruction from the ECU 11. That is, the steering control device 13, the driving force control device 14, and the braking force control device 15 cooperate with each other to enable automatic driving of the vehicle 30.

The ADAS system mounted on the vehicle 30 is not limited to the device described above, but another device may be mounted thereon.

The sensor controller 21 is connected to surrounding cameras 21a, driver monitor cameras 21b, range sensors 21c, etc., and passes sensor outputs thereof to the ECU 11. The ECU 11 senses surrounding environment of the vehicle 30, and detects a biological signal of the driver on the basis of acquired information. The sensors connected to the sensor controller 21 are not limited to these examples, but another sensor may be connected thereto.

The surrounding cameras 21a are installed to be oriented toward different directions around the vehicle 30 to acquire image information of surroundings of the vehicle 30.

The driver monitor camera 21b is installed in an instrument panel of the vehicle 30 to acquire an image including a face of the driver during driving. The driver monitor camera 21b may be installed at feet of the driver to monitor an accelerator operation or a brake operation performed by the driver.

The range sensors 21c are installed to be oriented toward different directions around the vehicle 30 to measure a distance to an obstacle around the vehicle 30. The range sensor 21c is, for example, an ultrasonic sensor that measures a short distance, a millimeter wave radar that measures a middle or long distance, Light Detection and Ranging (LiDAR), and so forth.

The GPS receiver 22 acquires, through the GPS antenna 23, GPS signals transmitted from Global Positioning System (GPS) satellites to measure a current position and a driving direction of the vehicle 30. The ECU 11 specifies a road on which the vehicle 30 is driving and a driving direction thereof by comparing the specified current position and driving direction of the vehicle 30 with the map database 24 (map matching). A method of specifying the current position and the driving direction of the vehicle using GPS signals and the map database has been widely put to practical use in car navigation systems, so that detailed description thereof will not be provided.

The display device 25 displays information such as information related to a driving state of the vehicle 30, information presented to the driver, etc. The display device 25 includes, for example, a center monitor 25a, an indicator 25b, a meter 25c, and so forth illustrated in FIG. 4. Contents of the display device 25 will be described later (refer to FIG. 4). The display device 25 may be a device, such as a speaker or a vibration device, which presents information for a sense of hearing or a sense of touch in addition to a sense of sight of the driver.

The operation device 26 acquires various kinds of operation information for the vehicle 30. The operation device 26 is, for example, a touch panel laminated on a display surface of the center monitor 25a, a physical switch disposed on the instrument panel, etc.

The communication interface 27 connects the vehicle 30 and a portable terminal (for example, a smartphone) outside the vehicle by wireless communication. The communication interface 27 transmits, from the vehicle 30 to the portable terminal, the evaluation score E of the cognitive function calculated by the driving characteristic determination device 10, for example.

Next, with reference to FIG. 4, the following describes a schematic configuration of the cockpit of the vehicle 30 on which the driving characteristic determination device 10 is mounted.

The center monitor 25a as an example of the display device 25 is installed in a center cluster of the vehicle 30. To improve visibility during driving, the center monitor is disposed at the highest possible place. The driving characteristic determination device 10 displays the evaluation score E of the cognitive function, a driving assist content based on the evaluation score E, etc., on the center monitor 25a.

The indicator 25b as an example of the display device 25 is disposed at an upper end of a spoke of a steering wheel 31 along the upper end. The indicator 25b is formed of a rod-shaped light guide body, for example, and emits light in color corresponding to incident light that is incident from one end. The driving characteristic determination device 10 causes the indicator 25b to emit light in color corresponding to a driving assist content based on the evaluation score E of the cognitive function. The indicator is disposed in a peripheral vision region of the driver during driving, and a light emission color of the indicator 25b can be recognized without turning eyes on the indicator 25b. Due to this, the driver can easily recognize the driving assist content.

The meter 25c as an example of the display device 25 is disposed in a meter cluster of the vehicle 30. The meter 25c is, for example, a speedometer, an engine speed meter, a fuel gauge, a water-temperature gauge, and so forth.

Additionally, the driver monitor camera 21b is installed in the meter cluster of the vehicle 30. The driver monitor camera 21b is disposed in the meter cluster so as to completely image a region where there are eyeballs of the driver during driving (eye range).

Functional Configuration of Driving Characteristic Determination Device

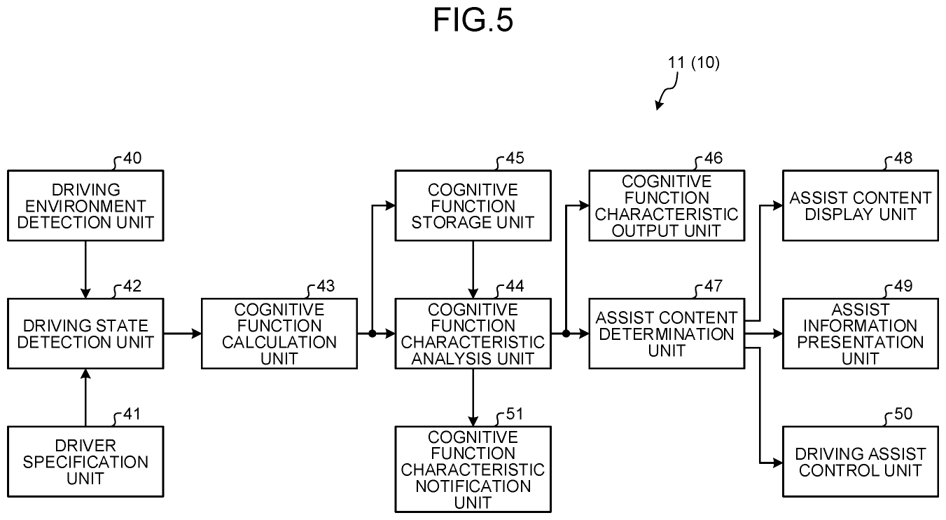

Next, with reference to FIG. 5, the following describes a functional configuration of the driving characteristic determination device 10. FIG. 5 is a functional block diagram illustrating an example of the functional configuration of the driving characteristic determination device according to the embodiment.

By loading control programs stored in the ECU 11 into the RAM 11b to be operated by the CPU 11a, the ECU 11 of the driving characteristic determination device 10 implements, as functional units, a driving environment detection unit 40, a driver specification unit 41, a driving state detection unit 42, a cognitive function calculation unit 43, a cognitive function characteristic analysis unit 44, a cognitive function storage unit 45, a cognitive function characteristic output unit 46, an assist content determination unit 47, an assist content display unit 48, an assist information presentation unit 49, a driving assist control unit 50, and a cognitive function characteristic notification unit 51 illustrated in FIG. 5.

The driving environment detection unit 40 detects a state of surrounding environment of the road on which the vehicle 30 is driving. The state of the surrounding environment of the road is, for example, information about a road shape in front of the driving direction, the number of lanes, a speed limit, a distance to an intersection, a shape of an intersection, presence/absence of a preceding vehicle and an inter-vehicle distance, presence/absence of an oncoming vehicle and a presence position, presence/absence of a pedestrian and a presence position, etc. These pieces of information can be obtained by, for example, analyzing an image taken by the surrounding camera 21a and information acquired by the range sensor 21c, and making comparison between a current position of the vehicle 30 acquired from GPS signals and the map database 24.

The driver specification unit 41 specifies a driver who is driving the vehicle 30. For example, the driver specification unit 41 specifies the driver who is currently driving by comparing a face image of the driver taken by the driver monitor camera 21b with a face image of a driver registered in advance. In a case where no comparison result is obtained, the driver is instructed to perform a new registration for him/her as a new driver. The driver specification unit 41 is an example of a specification unit in the present disclosure.

The driving state detection unit 42 detects at least one of driving behavior for the vehicle 30 performed by the driver, biological information of the driver during driving, and behavior of the vehicle 30.

The cognitive function calculation unit 43 calculates the evaluation score E indicating whether the cognitive function of the driver is high or low, on the basis of the information detected by the driving state detection unit 42. The evaluation score E is an example of a numerical value in the present disclosure.

The cognitive function characteristic analysis unit 44 analyzes the evaluation score E of the cognitive function calculated by the cognitive function calculation unit 43. The evaluation score E is analyzed as cognitive function characteristics relative to one or more different brain functions. The cognitive function characteristics relative to one or more different brain functions are, for example, the ability for memory 80, the ability for executive function 81, the ability for attention 82, the ability for information processing 83, the ability for visual-spatial cognition 84, etc. described above.

The cognitive function storage unit 45 stores, in association with the driver, the evaluation score E of the cognitive function calculated by the cognitive function calculation unit 43.

The cognitive function characteristic output unit 46 outputs information about an analysis result obtained by the cognitive function characteristic analysis unit 44. The cognitive function characteristic output unit 46 is an example of an output unit in the present disclosure.

The assist content determination unit 47 determines whether to enable a function of assisting information presentation for suppressing further decline of the cognitive function characteristic of the driver, or enable a function of assisting a driving operation relative to the cognitive function characteristic, from among functions of the vehicle 30. The determination by the assist content determination unit 47 is performed on the basis of comparison between a threshold and the cognitive function characteristic calculated by the cognitive function characteristic analysis unit 44. The assist content determination unit 47 is an example of a determination unit in the present disclosure.

The assist content display unit 48 displays an assist content determined by the assist content determination unit 47 on the center monitor 25a, for example.

In a case where the assist content determination unit 47 determines to enable the function of assisting information presentation for suppressing further decline of the cognitive function characteristic of the driver, the assist information presentation unit 49 performs the information presentation. In the following description, a mode of enabling the function of assisting information presentation for suppressing further decline of the cognitive function characteristic of the driver is referred to as a training mode.

In a case where the assist content determination unit 47 determines to enable the function of assisting a driving operation relative to the cognitive function characteristic, the driving assist control unit 50 activates the function. In the following description, a mode of enabling the function of assisting a driving operation relative to the cognitive function characteristic is referred to as a driving assist mode.

The cognitive function characteristic notification unit 51 makes notification about temporal changes of the evaluation score E of the cognitive function of the same driver. The cognitive function characteristic notification unit 51 is an example of a notification unit in the present disclosure.

Operation of Driving State Detection Unit

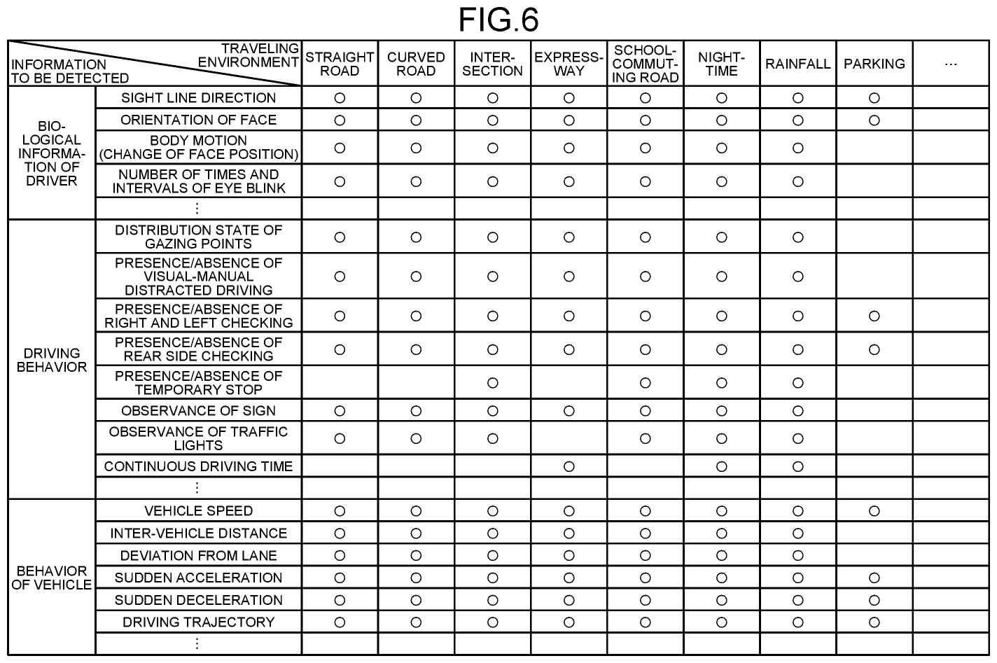

With reference to FIG. 6, the following describes a detailed effect of the driving state detection unit 42. FIG. 6 is a diagram for explaining an example of information (an example of the driving information) to be detected by the driving state detection unit.

The driving state detection unit 42 detects biological information of the driver by analyzing an image including a face of the driver taken by the driver monitor camera 21b illustrated in FIG. 3. Specifically, the driving state detection unit 42 detects a gaze direction of the driver, orientation of the face, body motion (change of a face position), the number of times and intervals of eye blink, and so forth. The biological information to be detected and the detection method are not limited to the content described above. For example, heartbeat, a body temperature, a respiration state, etc. of the driver may be detected. As a specific method for detecting a state of the driver, vehicle information, operation information, and biological information, the method summarized in “Ryoko Fukuda, Fumio Harada, Taisaku Okumura: “Vehicle for a Super-Aged Society: Focusing on One's Way of Being”, Cognitive Studies, 25(3), pp. 259-278, 2018. 09” may be used, or another method may be used.

The driving state detection unit 42 also detects behavior of the vehicle 30 on the basis of outputs from the accelerator position sensor 12a, the brake pedal stepping force sensor 12b, the steering angle sensor 12c, and the range sensor 21c illustrated in FIG. 3, and outputs from various sensors (a vehicle speed sensor, a shift position sensor, etc.) included in the vehicle 30 that are not illustrated in FIG. 3. Specifically, the driving state detection unit 42 detects behavior of the vehicle 30 such as a vehicle speed, an inter-vehicle distance, whether the vehicle is departed from the lane, sudden acceleration, sudden deceleration, a driving trajectory, and so forth. As a method for measuring vehicle behavior such as displacement of a vehicle position with respect to a road, displacement of a steering angle, and a pedal reaction time, the method described in “Shinya Takagi, Keiichi Yamada: “On the Relationship between Behavior of a Vehicle and Reaction Time of the Driver”, Transactions of the Society of Automotive Engineers of Japan, Vol. 43, No. 5, pp. 1131-1137, 2012” may be used, or another method may be used. As a method for measuring an inter-vehicle distance, the method described in “Li Bo, Zhang Xiaolin, Makoto Sato: “Pitch Angle Estimation Using a Vehicle Mounted Monocular Camera for Vehicle Target Range Measurement”, The journal of the Institute of Image Information and Television Engineers, vol. 69, No. 4, pp. J169-J176, 2015” may be used, or information detected by a typical ADAS system may be used to implement the method. The behavior of the vehicle 30 to be detected is not limited to the content described above.

The driving state detection unit 42 detects driving behavior of the driver on the basis of the biological information of the driver, the behavior of the vehicle 30, and the road environment in which the vehicle 30 is driving that have been detected. Specifically, the driving state detection unit 42 detects driving behavior such as a distribution state of gazing points, presence/absence of visual-manual distracted driving, presence/absence of right and left checking, presence/absence of rear side checking, presence/absence of temporary stop, observance of a traffic sign, observance of traffic lights, a continuous driving time, and so forth. The driving behavior of the driver to be detected is not limited to the content described above.

The distribution state of gazing points can be obtained by analyzing the measured gaze direction. The gazing point is a point at which the gaze direction is retained for a predetermined time or more. In a case where the gazing points are distributed in a wide range, it is estimated that the driver is paying attention to the wide range. On the other hand, in a case where the gazing points concentrate in a narrow range, it is estimated that the attention of the driver is attracted by a specific range. As a method for detecting orientation of a gaze, for example, the method described in “Tatsuki Kamisaka, Masafumi Noda, Yoshito Mekada, Daisuke Deguchi, Ichiro Ide, Hiroshi Murase: “Prediction of driving behavior using driver's gaze information”, IEICE technical report, MI, medical image 111 (49), 105-110, 2011-05-12” or “Akito Yamasaki, Pongsathorn Raksincharoensak, Motoki Shino: “Extraction of Driver's Gaze Region by Face Direction Estimation Using On-Board Cameras”, Transactions of the Society of Automotive Engineers of Japan, Vol. 48, No. 5, pp. 1113-1119, 2017” may be used, or another method may be used.

The presence/absence of visual-manual distracted driving can be obtained by analyzing the measured gaze direction and orientation of the face. As a method for detecting the presence/absence of visual-manual distracted driving, for example, the method described in “Akito Yamasaki, Pongsathorn Raksincharoensak, Motoki Shino: “Extraction of Driver's Gaze Region by Face Direction Estimation Using On-Board Cameras”, Transactions of the Society of Automotive Engineers of Japan, Vol. 48, No. pp. 1113-1119, 2017” may be used, or another method may be used.

The presence/absence of right and left checking can be checked by determining whether orientation of the face has moved to the left or right, or whether the gaze is directed toward a direction in which safety should be confirmed at a place where right and left checking should be performed. The place where right and left checking should be performed can be determined by specifying that the vehicle is driving in front of the intersection where right and left checking is required, for example, by comparing the current position of the vehicle 30 acquired from GPS signals with the map database 24. For example, whether pedestrians are checked may be detected by using the technique described in “Akito Yamasaki, Pongsathorn Raksincharoensak, Motoki Shino: “Extraction of Driver's Gaze Region by Face Direction Estimation Using On-Board Cameras”, Transactions of the Society of Automotive Engineers of Japan, Vol. 48, No. pp. 1113-1119, 2017”, for example, or another method may be used.

The presence/absence of rear side checking can be checked by determining whether the face is oriented rearward or oriented in a direction of a room mirror or a rearview mirror at a place where rear side checking should be performed. The presence/absence of rear side checking may be checked by using the technique described in “Akito Yamasaki, Pongsathorn Raksincharoensak, Motoki Shino: “Extraction of Driver's Gaze Region by Face Direction Estimation Using On-Board Cameras”, Transactions of the Society of Automotive Engineers of Japan, Vol. 48, No. 5, pp. 1113-1119, 2017”, for example, or another method may be used. The place where rear side checking should be performed may be estimated when a shift position of the vehicle 30 is switched to a reverse position, for example.

The presence/absence of temporary stop can be checked by determining whether the vehicle 30 has stopped at a place where temporary stop should be performed. The place where the temporary stop should be performed can be specified when the surrounding camera 21a detects a traffic sign of temporary stop. As a method for recognizing the traffic sign, for example, the method described in “Masanari Takagi, Hironobu Fujiyoshi: “Traffic Sign Recognition Using SIFT features”, IEEJ Transactions on Electronics, Information and Systems, vol. 129, No. 5, pp. 824-831, 2009” may be used, or another method may be used.

Observance of the traffic sign can be determined by checking whether the content of the traffic sign detected by the surrounding camera 21a matches the detected behavior of the vehicle 30.

Observance of traffic lights can be determined by checking whether a state of traffic lights detected by the surrounding camera 21a matches the detected behavior of the vehicle 30.

The continuous driving time can be specified on the basis of an elapsed time after an ignition is turned ON, for example.

The driving environment of the vehicle 30 continuously changes. It is not preferable to continuously detect the detection targets described above because a load on a calculator increases. Therefore, the driving state detection unit 42 detects, on the basis of the driving environment of the vehicle 30, at least one of the driving behavior for the vehicle 30 by the driver, the biological information of the driver during driving, and the behavior of the vehicle 30 that are expected to be caused in the driving environment.

Specifically, on the basis of the driving environment detected by the driving environment detection unit 40, the driving state detection unit 42 estimates the biological information, the behavior of the vehicle 30, and the driving behavior that are expected to be caused in the driving environment, and then detects only at least the estimated information to narrow down the detection target.

In horizontal rows in FIG. 6, examples of the driving environment detected by the driving environment detection unit 40 are listed. In vertical rows in FIG. 6, the detection targets described above are listed. Each circle depicted in FIG. 6 indicates the detection target that should be detected in the corresponding driving environment.

For instance, in a case where the vehicle 30 that is driving in front of an intersection is detected, the driving state detection unit 42 detects information related to the behavior of the driver that is expected to be caused at the intersection. In this case, a gaze direction and orientation of the face are detected as the biological information of this driver. Additionally, a vehicle speed, sudden acceleration, sudden deceleration, and a driving trajectory are detected as the behavior of the vehicle 30. Moreover, a distribution state of gazing points, presence/absence of right and left checking, presence/absence of temporary stop, observance of a traffic sign, and observance of traffic lights are detected as the driving behavior of the driver. The setting of the circles in FIG. 6 is merely examples, and the embodiment is not limited to the examples.

A calculation load increases if the detection target corresponding to the driving environment is estimated every time. Therefore, for example, the map illustrated in FIG. 6 may be stored in advance in the storage device 11d so that the driving state detection unit 42 can select the detection target with making reference to the stored map.

Method for Calculating Cognitive Function

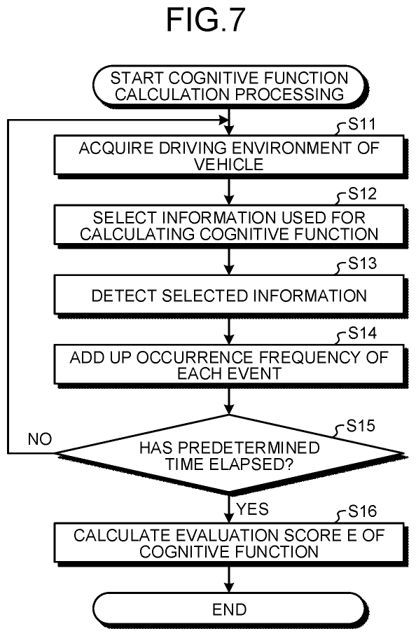

With reference to FIG. 7, the following describes a method for calculating the evaluation score E of the cognitive function by the cognitive function calculation unit 43. FIG. 7 is a flowchart illustrating an example of a processing procedure of calculating the evaluation score of the cognitive function by the cognitive function calculation unit.

The driving environment detection unit 40 detects the driving environment of the vehicle 30 (Step S11).

The driving state detection unit 42 selects information to be detected for calculating the cognitive function, on the basis of the driving environment detected by the driving environment detection unit 40 (Step S12).

The driving state detection unit 42 detects the information selected at Step S12 (Step S13).

On the basis of the information detected by the driving state detection unit 42, the cognitive function calculation unit 43 adds up, for each event, an occurrence frequency of the event that matches the driving environment detected by the driving environment detection unit 40 (Step S14).

The cognitive function calculation unit 43 determines whether a predetermined time has elapsed (Step S15). In response to determining that the predetermined time has elapsed (Yes at Step S15), the process proceeds to Step S16. On the other hand, if the predetermined time has not elapsed (No at Step S15), the process returns to Step S11. The predetermined time may be optionally set, and the determination is performed in units of one minute, for example.

In response to determining at Step S15 that the predetermined time has elapsed, the cognitive function calculation unit 43 calculates the evaluation score E of the cognitive function (Step S16). For example, the occurrence frequency of the event calculated at Step S14 is applied to the evaluation score E. The cognitive function calculation unit 43 then ends the processing in FIG. 7. Note that, for example, the distribution state of gazing points cannot be expressed by frequency, so that a numerical value representing a size of a distribution range may be applied to the evaluation score E. For each of other pieces of the information that cannot be represented by the frequency, the evaluation score E may be calculated on the basis of a calculation method that is set for each piece of the information.

Note that the occurrence frequency of the event is added up at Step S14, whereas subtraction may be performed on accumulated occurrence frequencies of the event in response to detecting that preferable driving behavior has been performed.

Analysis of Cognitive Function

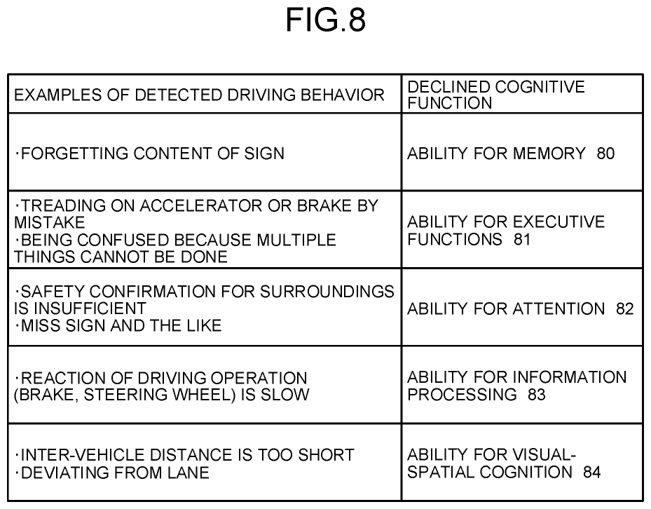

Next, with reference to FIG. 8, the following describes a method for analyzing the evaluation score E of the cognitive function by the cognitive function characteristic analysis unit 44. FIG. 8 is a diagram for explaining a relation between cognitive function characteristics relative to different brain functions and driving behavior that is caused during driving.

As illustrated in FIG. 8, the cognitive function characteristic analysis unit 44 analyzes a degree of decline for the respective cognitive functions relative to different brain functions, on the basis of a type of the detected driving behavior and the occurrence frequency thereof. Influence on driving due to decline of the cognitive functions is described in “Masaru Mimura, Yoshio Fujita “Automobile Driving and Cognitive Function About the Driving”, Japanese Journal of Geriatrics, vol. 55, No. 2, pp. 191-196, 2018”, “Shinya lida, Noriaki Kato, Kenji Hachisuka, Satoru Saeki: “Determination of driving ability of elderly people”, Japanese Journal of Geriatrics, vol. No. 2, pp. 202-207, 2018”, “Naoto Kamimura: “Driving Ability and Assessment of Fitness to Drive for Dementia/Cognitive Decline”, The Journal of the International Association of Traffic and Safety Sciences, vol. 42, No. 3, pp. 12-22, 2018”, and “Katsuya Urakami: “Dementia and driving”, Society of Automotive Engineers of Japan, Inc., vol. 71, No. 12, pp. 90-95, 2017”. Influence due to lowering of information processing speed is described in “David W. Eby, Lisa J. Molnar, Paula S. Kartje (Edited and translated by Etsuo Horikawa, Tomoko Mine): “Maintaining safe mobility in an aging society”, Kyoto University Press, pp. 15-33, 2020” and “Tsuneo Matsuura: “Safety psychology of elderly driver”, University of Tokyo Press, pp. 48-62, 2017”. The driving behaviors illustrated in FIG. 8 are merely examples, and a table of correspondence that is different therefrom may be used.

For instance, when the ability for memory 80 declines, it becomes difficult to retain information written in a traffic sign, or the driver forgets where to go and gets lost (Shinya lida, Noriaki Kato, Kenji Hachisuka, Satoru Saeki: “Determination of driving ability of elderly people”, Japanese Journal of Geriatrics, vol. 55, No. 2, pp. 202-207, 2018), or the driver forgets past experiences such as collision of a car or embarrassment (Naoto Kamimura: “Driving Ability and Assessment of Fitness to Drive for Dementia/Cognitive Decline”, The Journal of the International Association of Traffic and Safety Sciences, vol. 42, No. 3, pp. 12-22, 2018). The driver may be disabled from understanding a traffic sign or a traffic law (Masaru Mimura, Yoshio Fujita “Automobile Driving and Cognitive Function About the Driving”, Japanese Journal of Geriatrics, vol. 55, No. 2, pp. 191-196, 2018). The cognitive function characteristic analysis unit 44 calculates an evaluation score Ea of the ability for memory 80 from evaluation scores E calculated by the cognitive function calculation unit 43, on the basis of a frequency of observing a traffic sign, a frequency of observing traffic lights, etc. As a method for recognizing a traffic sign, for example, the method described in “Masanari Takagi, Hironobu Fujiyoshi: “Traffic Sign Recognition Using SIFT features”, IEEJ Transactions on Electronics, Information and Systems, vol. 129, No. 5, pp. 824-831, 2009” may be used, or another method may be used. It may be determined that the driver has recognized content of the traffic sign on the basis of whether the driver has performed driving behavior matching the content of the traffic sign.

When the ability for executive function 81 declines, the driver may step on an accelerator or a brake pedal by mistake, or it becomes difficult to perform a plurality of pieces of information processing (Shinya Iida, Noriaki Kato, Kenji Hachisuka, Satoru Saeki: “Determination of driving ability of elderly people”, Japanese Journal of Geriatrics, vol. 55, No. 2, pp. 202-207, 2018). Additionally, the driver may be disabled from determining action that should be performed next when a planned route is unavailable (Naoto Kamimura: “Driving Ability and Assessment of Fitness to Drive for Dementia/Cognitive Decline”, The Journal of the International Association of Traffic and Safety Sciences, vol. 42, No. 3, pp. 12-22, 2018), or disabled from taking a measure appropriate to a situation (Masaru Mimura, Yoshio Fujita “Automobile Driving and Cognitive Function About the Driving”, Japanese Journal of Geriatrics, vol. 55, No. 2, pp. 191-196, 2018). In some cases, the driver may be disabled from operating a car navigation system (Naoto Kamimura: “Driving Ability and Assessment of Fitness to Drive for Dementia/Cognitive Decline”, The Journal of the International Association of Traffic and Safety Sciences, vol. 42, No. 3, pp. 12-22, 2018). The cognitive function characteristic analysis unit 44 calculates an evaluation score Eb of the ability for executive function 81 from the evaluation scores E calculated by the cognitive function calculation unit 43, on the basis of the occurrence frequency of sudden acceleration and sudden deceleration.

When the ability for attention 82 declines, the driver is disabled from paying attention to the surrounding environment such as a traffic sign or traffic lights (Shinya lida, Noriaki Kato, Kenji Hachisuka, Satoru Saeki: “Determination of driving ability of elderly people”, Japanese Journal of Geriatrics, vol. 55, No. 2, pp. 202-207, 2018). The driver may miss traffic lights, or fails to notice an oncoming person (Naoto Kamimura: “Driving Ability and Assessment of Fitness to Drive for Dementia/Cognitive Decline”, The Journal of the International Association of Traffic and Safety Sciences, vol. 42, No. 3, pp. 12-22, 2018). The driver may be disabled from paying attention to surroundings at the time of changing a lane and perform a dangerous operation, or fails to notice a pedestrian or a motorcycle at the time of turning to the right or left (Shinya lida, Noriaki Kato, Kenji Hachisuka, Satoru Saeki: “Determination of driving ability of elderly people”, Japanese Journal of Geriatrics, vol. 55, No. 2, pp. 202-207, 2018). When the driver's attention is distracted, the driver pays attention to an occurrence inside the vehicle or outside the vehicle (David W. Eby, Lisa J. Molnar, Paula S. Kartje (Edited and translated by Etsuo Horikawa, Tomoko Mine): “Maintaining safe mobility in an aging society”, Kyoto University Press, pp. 15-33, 2020), and visual-manual distracted driving is caused. The cognitive function characteristic analysis unit 44 calculates an evaluation score Ec of the ability for attention 82 from the evaluation scores E calculated by the cognitive function calculation unit 43 on the basis of the distribution state of gazing points, a frequency of observing a traffic sign, a frequency of observing traffic lights, and so forth. As a method for detecting orientation of a gaze, for example, the method described in “Tatsuki Kamisaka, Masafumi Noda, Yoshito Mekada, Daisuke Deguchi, Ichiro Ide, Hiroshi Murase: “Prediction of driving behavior using driver's gaze information”, IEICE technical report, MI, medical image 111 (49), 105-110, 2011-05-12” or “Akito Yamasaki, Pongsathorn Raksincharoensak, Motoki Shino: “Extraction of Driver's Gaze Region by Face Direction Estimation Using On-Board Cameras”, Transactions of the Society of Automotive Engineers of Japan, Vol. 48, No. 5, pp. 1113-1119, 2017” may be used, and it is possible to evaluate whether the driver is seeing a point that should be watched such as a traffic sign or a pedestrian on the basis of movement of the gaze. Alternatively, the evaluation score Ec of the ability for attention 82 may be calculated by assigning weights to the evaluation scores E respectively calculated for whether safety confirmation for the surroundings is insufficient and whether the driver misses a traffic sign in driving behavior examples illustrated in FIG. 8. As a weighting coefficient, a coefficient determined in advance may be used, or a correlation with the cognitive function may be sequentially learned.

When the ability for information processing 83 declines, it takes more time to find danger on a congested road or a road on which vehicles travel fast, and coping with the danger may be delayed (Tsuneo Matsuura: “Safety psychology of elderly driver”, University of Tokyo Press, pp. 48-62, 2017). Additionally, stop-and-go driving, hesitating driving, and unexpected operation error are increased (David W. Eby, Lisa J. Molnar, Paula S. Kartje (Edited and translated by Etsuo Horikawa, Tomoko Mine): “Maintaining safe mobility in an aging society”, Kyoto University Press, pp. 15-33, 2020). The cognitive function characteristic analysis unit 44 calculates an evaluation score Ed of the ability for information processing 83 from the evaluation scores E calculated by the cognitive function calculation unit 43, on the basis of a reaction time of a brake as a driving operation. For example, a brake timing is evaluated and calculated by using the method described in “Kazuyoshi Isaji, Naohiko Tsuru, Takahiro Wada, Shun'ichi Doi, Hiroshi Kaneko: “Analysis of the Brake Initiation Timing based on Performance Index for Approach and Alienation”, Transactions of the Society of Automotive Engineers of Japan, vol. 41, No. 3, pp. 593-598, 2010”.

When the ability for visual-spatial cognition 84 declines, a sense of distance to a preceding vehicle may become disordered, or the vehicle may depart from a lane when going round a curve (Shinya lida, Noriaki Kato, Kenji Hachisuka, Satoru Saeki: “Determination of driving ability of elderly people”, Japanese Journal of Geriatrics, vol. No. 2, pp. 202-207, 2018). Additionally, it becomes difficult to grasp a relation between an object and a size of the driver's own vehicle (Katsuya Urakami: “Dementia and driving”, Society of Automotive Engineers of Japan, Inc., vol. 71, No. 12, pp. 90-2017). The cognitive function characteristic analysis unit 44 calculates an evaluation score Ee of the ability for visual-spatial cognition 84 from the evaluation scores E calculated by the cognitive function calculation unit 43, on the basis of an average value of the inter-vehicle distance, the number of times of lane departure, and so forth. As a method for measuring the vehicle behavior such as a displacement of a vehicle position with respect to a road, displacement of a steering angle, a pedal reaction time, etc., the method described in “Shinya Takagi, Keiichi Yamada: “On the Relationship between Behavior of a Vehicle and Reaction Time of the Driver”, Transactions of the Society of Automotive Engineers of Japan, Vol. 43, No. 5, pp. 1131-1137, 2012” can be used. As a method for measuring the inter-vehicle distance, the method described in “Li Bo, Zhang Xiaolin, Makoto Sato: “Pitch Angle Estimation Using a Vehicle Mounted Monocular Camera for Vehicle Target Range Measurement”, The journal of the Institute of Image Information and Television Engineers, vol. 69, No. 4, pp. J169-J176, 2015” can be used, and the inter-vehicle distance can also be calculated by using information detected by a typical ADAS system.

It is efficient that the evaluation scores Ea, Eb, Ec, Ed, and Ee of the respective cognitive functions are calculated on the basis of, for example, a table prepared in advance that indicates correspondence between a detection result of an operation state and the evaluation scores Ea, Eb, Ec, Ed, and Ee.

The cognitive function characteristic analysis unit 44 evaluates a degree of each of the cognitive functions of the driver by comparing the evaluation scores Ea, Eb, Ec, Ed, and Ee calculated as described above with the first threshold Th1 and the second threshold Th2 described above.

In a case where each of the evaluation scores Ea, Eb, Ec, Ed, and Ee is larger than the first threshold Th1, the driving characteristic determination device 10 according to the present embodiment determines that the cognitive function of the driver is in a normal state, that is, a safe state. In a case where each of the evaluation scores Ea, Eb, Ec, Ed, and Ee is smaller than the first threshold Th1 and larger than the second threshold Th2, the driving characteristic determination device 10 determines that the corresponding cognitive function is in a state of requiring attention in which attention is required for driving. Moreover, in a case where each of the evaluation scores Ea, Eb, Ec, Ed, and Ee is smaller than the second threshold Th2, the driving characteristic determination device 10 determines that the corresponding cognitive function is in a dangerous state.

The cognitive function characteristic analysis unit 44 may analyze only the cognitive function that is calculated by the cognitive function calculation unit 43 at the present time, or may analyze it together with a past cognitive function that is stored by the cognitive function storage unit 45 in association with the driver. By analyzing the cognitive function together with the past cognitive function, it can be estimated whether the cognitive function is on a recovery trend or a decline trend. The training mode may be positively activated with respect to the cognitive function on a recovery trend. In a case where a long-term decline trend of the cognitive function is shown, the training mode may be activated to prevent further decline.

Depending on the driving environment of the vehicle 30, there is a case where an event to be analyzed by the cognitive function calculation unit 43 and the cognitive function characteristic analysis unit 44 does not constantly occur. Thus, all the evaluation scores Ea, Eb, Ec, Ed, and Ee related to all the cognitive functions as targets are not necessarily obtained at the same time.

Method for Determining Content of Assist Corresponding to Evaluation Score of Cognitive Function

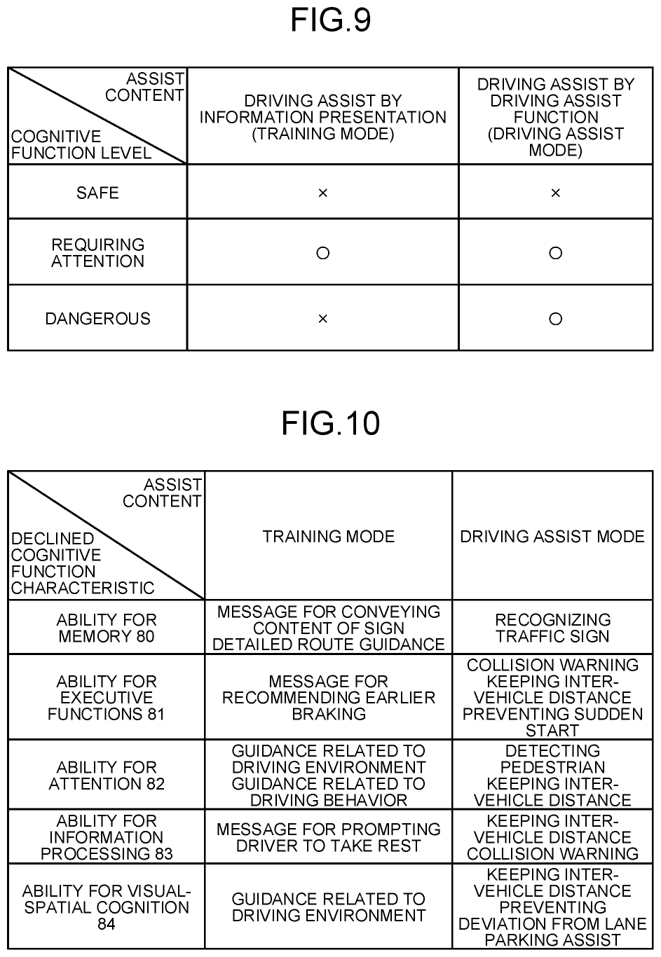

Next, with reference to FIG. 9 and FIG. 10, the following describes a method for determining content of assist performed by the driving characteristic determination device in accordance with the cognitive function characteristic. FIG. 9 is a first diagram for explaining an example of content of assist performed by the driving characteristic determination device in accordance with the cognitive function characteristic. FIG. 10 is a second diagram for explaining an example of content of assist performed by the driving characteristic determination device in accordance with the cognitive function characteristic.

As illustrated in FIG. 9, in a case where the driver is in a state of requiring attention to driving (at a level of requiring attention), the assist content determination unit 47 assists information presentation for suppressing further decline of the cognitive function of the driver. In short, a driving assist (training mode) by information presentation is activated. This is because the cognitive function of the driver is not in a completely declined state, so that there is the possibility that the declined cognitive function can be recovered to a normal level by causing driving to be continued while performing training related to the corresponding cognitive function. For example, regarding a temporal cognitive function, the cognitive function is expected to be recovered while receiving the driving assist. In a case of a state where the cognitive function chronically declines that is called mild cognitive impairment (MCI) as a preliminary stage of dementia, there is the possibility that the cognitive function can be recovered due to such training. By causing the cognitive function required for driving the vehicle to be recovered by the training mode, the driver can be expected to continue safe driving.

As illustrated in FIG. 9, in a case where the cognitive function of the driver is at a dangerous level, the assist content determination unit 47 causes a function of assisting the corresponding cognitive function to operate, out of driving assist functions of the vehicle 30. That is, a driving assist (driving assist mode) by the driving assist function is activated.

The driving characteristic determination device 10 evaluates states of the cognitive function characteristics, and there is the possibility that those cognitive functions are each determined to be at the level of requiring attention. In such a case, the assist content determination unit 47 determines a cognitive function for which the training mode is enabled, and determines a cognitive function(s) for which the driving assist mode is enabled. Note that the assist content determination unit 47 enables the training mode for only one of the cognitive functions. This is because, if the training modes for the cognitive functions are activated at the same time, information to be presented to the driver increases, and thereby the driver may be confused. Therefore, the assist content determination unit 47 activates the driving assist mode for assisting one or more of the cognitive functions that are determined to be at the level of requiring attention. The one or more of cognitive functions are other than the cognitive function for which the training mode is activated. Moreover, in a case where the cognitive functions are determined to be at the dangerous level, the assist content determination unit 47 activates the driving assist mode related to the corresponding cognitive functions.

Next, with reference to FIG. 10, the following describes specific content of the training mode and the driving assist mode related to each of the cognitive functions.

When the ability for memory 80 declines to the level of requiring attention, the assist content determination unit 47 causes a function of recognizing content of a traffic sign and outputting a message for conveying the content, a function of giving detailed route guidance, etc. to operate as the training mode, for example. These functions assist recovery of the ability for memory 80 of the driver that is estimated to have declined. When the ability for memory 80 declines to the dangerous level, the assist content determination unit 47 causes a traffic sign recognition function of the vehicle 30 to operate, for example. An upper limit speed of the vehicle 30 may be set on the basis of content of a recognized traffic sign, for example, a speed limit. These functions can reduce careless mistakes caused by inattention.

When the ability for executive function 81 declines to the level of requiring attention, the assist content determination unit 47 causes a function of outputting a message recommending earlier braking, etc. to operate as the training mode. These functions assist recovery of the ability for executive function 81 of the driver that is estimated to have declined. When the ability for executive function 81 declines to the dangerous level, the assist content determination unit 47 causes, for example, a collision warning function, an inter-vehicle distance keeping function, a sudden start prevention function, or the like of the vehicle 30 to operate. These functions can assist execution of part of the driving operation performed by the driver.

When the ability for attention 82 declines to the level of requiring attention, the assist content determination unit 47 causes a function of outputting guidance related to driving environment or guidance related to driving behavior to operate as the training mode, for example. These functions assist recovery of the ability for attention 82 of the driver that is estimated to have declined. When the ability for attention 82 declines to the dangerous level, the assist content determination unit 47 causes a pedestrian detection function, an inter-vehicle distance keeping function and so forth of the vehicle 30 to operate, for example. These functions can cause the vehicle 30 to pay attention to part of a region to which the driver should pay attention.

When the ability for information processing 83 declines to the level of requiring attention, the assist content determination unit 47 causes a function of prompting the driver to focus on and execute one thing because driving assist is given to things other than the thing executed by the driver, or outputting a message for prompting the driver to take a rest, etc. to operate as the training mode, for example. These functions assist recovery of the ability for information processing 83 of the driver that is estimated to have declined. When the ability for information processing 83 declines to the dangerous level, the assist content determination unit 47 causes an inter-vehicle distance keeping function, a collision warning, etc. of the vehicle 30 to operate, for example. These functions can cause the vehicle 30 to perform part of information processing that should be performed by the driver.

When the ability for visual-spatial cognition 84 declines to the level of requiring attention, the assist content determination unit 47 causes a function of outputting guidance related to driving environment to operate as the training mode, for example. These functions assist recovery of the ability for visual-spatial cognition 84 of the driver that is estimated to have declined. When the ability for visual-spatial cognition 84 declines to the dangerous level, the assist content determination unit 47 causes an inter-vehicle distance keeping function, a lane departure preventing function, a parking assist function and so forth of the vehicle 30 to operate. These functions can cause the vehicle 30 to perform part of visual-spatial cognition that should be performed by the driver.

The driving characteristic determination device 10 continuously calculates the cognitive function even in a case where various assist modes are being activated. In a case where the cognitive function is recovered to a normal level, operation of the activated assist mode is stopped. The assist mode executed by the vehicle 30 is presented to the driver in a readily understandable form as described later.

Specific Method for Determining Assist Content

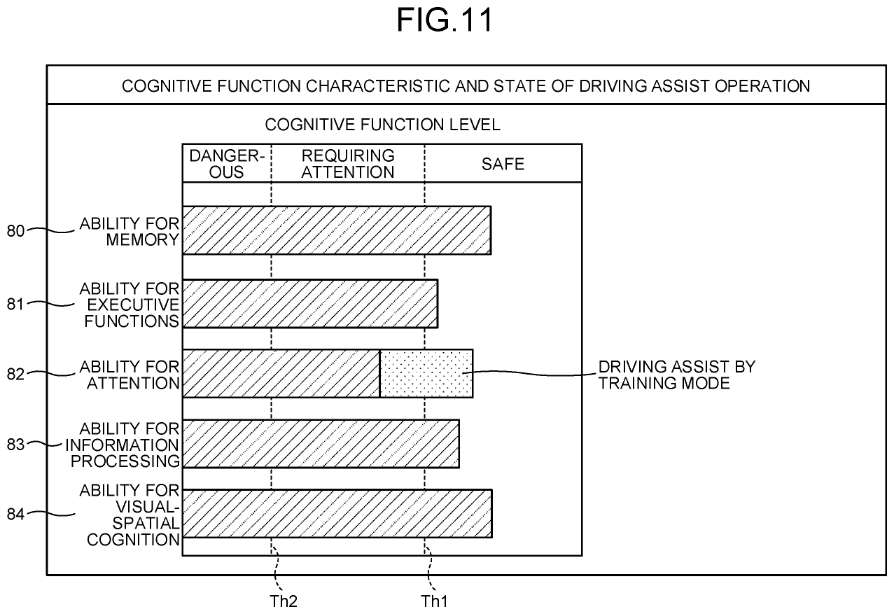

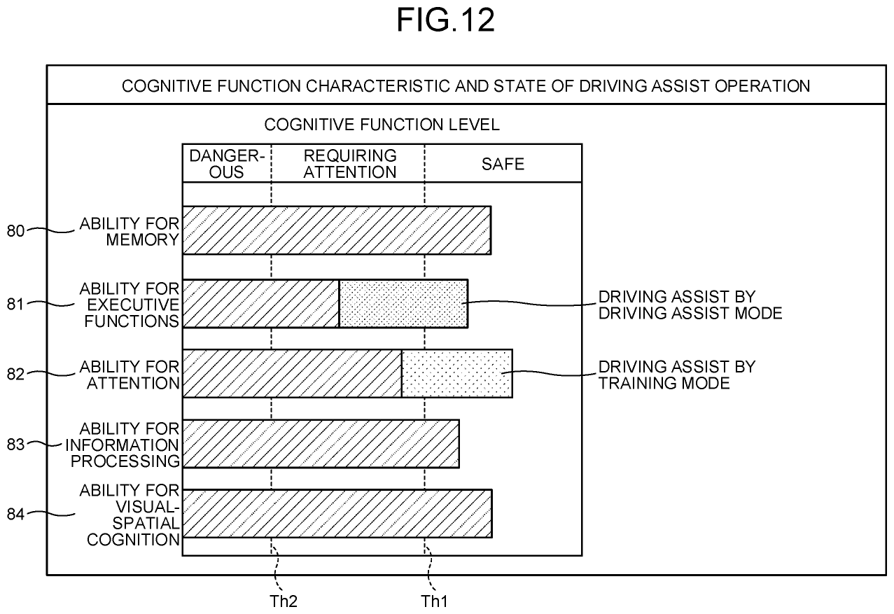

Next, with reference to FIG. 11 and FIG. 12, the following exemplifies a specific method for determining assist content. FIG. 11 is a first diagram for explaining a specific method for selecting a function of assisting the driver when the cognitive function characteristic declines. FIG. 12 is a second diagram for explaining a specific method for selecting a function of assisting the driver when the cognitive function characteristic declines.

FIG. 11 is an example of a case where the cognitive function characteristic analysis unit 44 determines that the evaluation score Ec of the ability for attention 82 of the driver is between the first threshold Th1 and the second threshold Th2, that is, at the level of requiring attention, and determines that the other cognitive functions are safe. At this point, the assist content determination unit 47 determines to activate the training mode related to the ability for attention 82. When the driver continues driving while executing the training mode related to the ability for attention 82, recovery of the ability for attention 82 is assisted. Specific content of the training mode will be described later.

FIG. 12 is an example of a case where the cognitive function characteristic analysis unit 44 determines that both of the evaluation score Eb of the ability for executive function 81 and the evaluation score Ec of the ability for attention 82 of the driver are between the first threshold Th1 and the second threshold Th2, that is, at the level of requiring attention, and determines that the other cognitive functions are safe. At this point, on the basis of a large/small relation between the evaluation score Eb and the evaluation score Ec, the assist content determination unit 47 determines to activate the training mode related to one cognitive function for the one cognitive function, and activate the driving assist mode related to the other cognitive function for the other cognitive function. In the example illustrated in FIG. 12, determination has been made such that the training mode is activated for the ability for attention 82 whose evaluation score is high, and the driving assist mode is activated for the ability for executive function 81 whose evaluation score is low. This is because, as the evaluation score of the cognitive function is higher, there is a higher possibility that the cognitive function is recovered by activating the training mode.

The cognitive function characteristic analysis unit 44 may classify the cognitive function into two or more levels on the basis of a calculation result of the cognitive function calculation unit 43. For example, the cognitive function may be classified into Level-1 indicating the cognitive function being high to Level-5 indicating the cognitive function being low. The assist content determination unit 47 may then determine the assist content on the basis of such a level of the cognitive function.

### Example of Information Presented to Driver

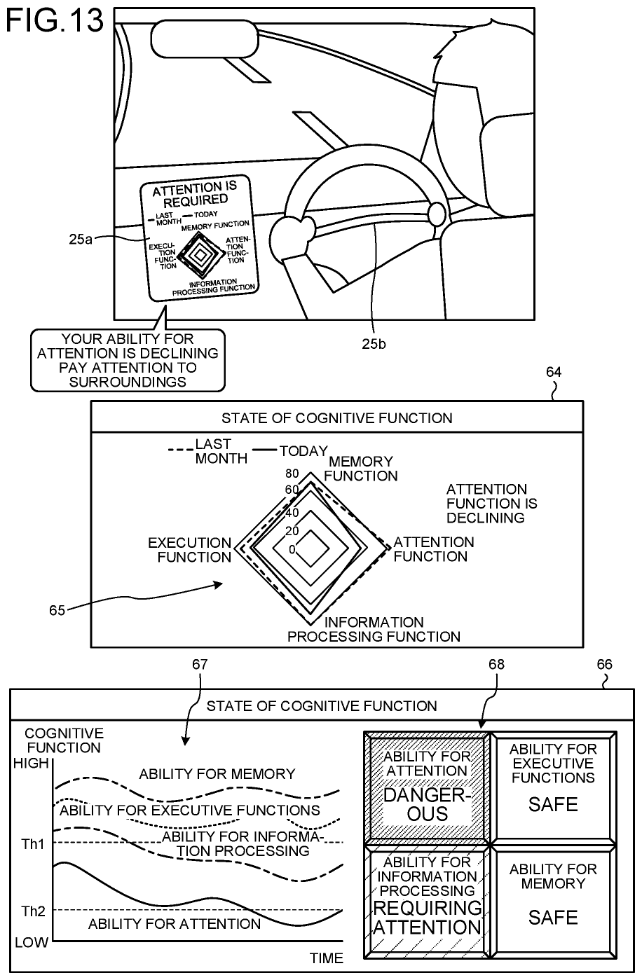

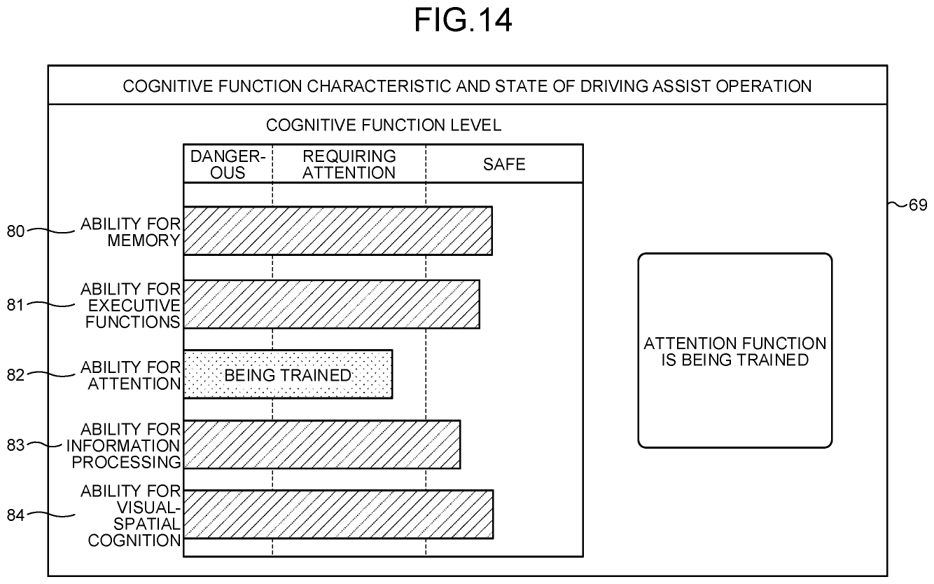

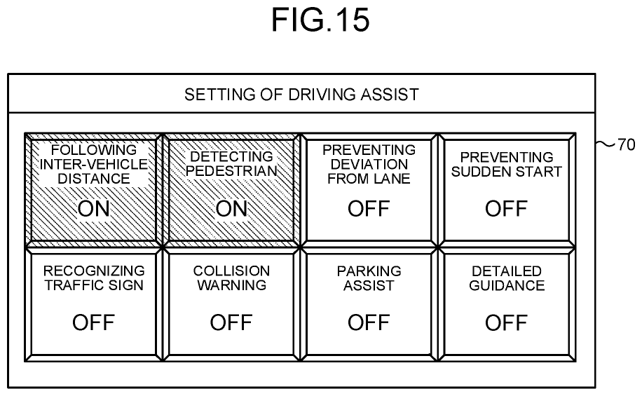

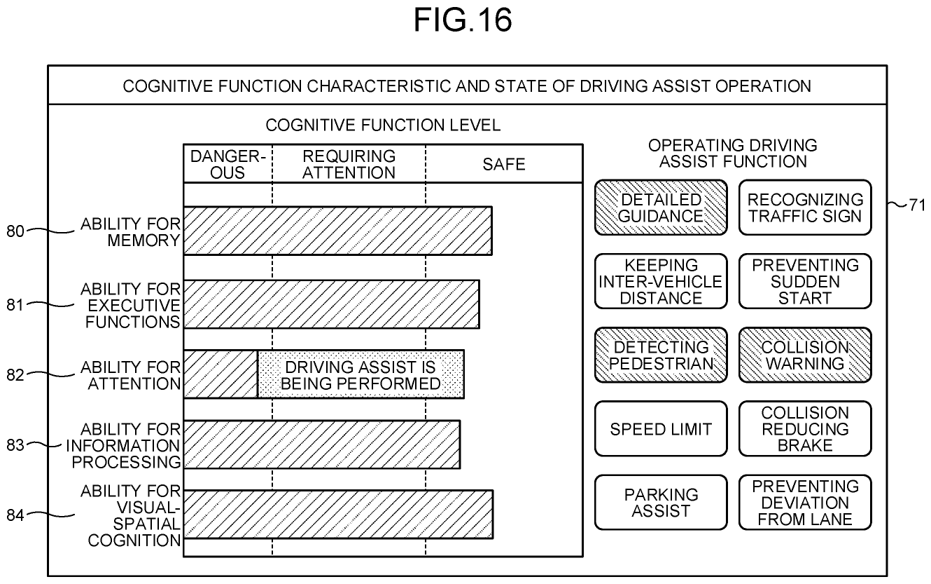

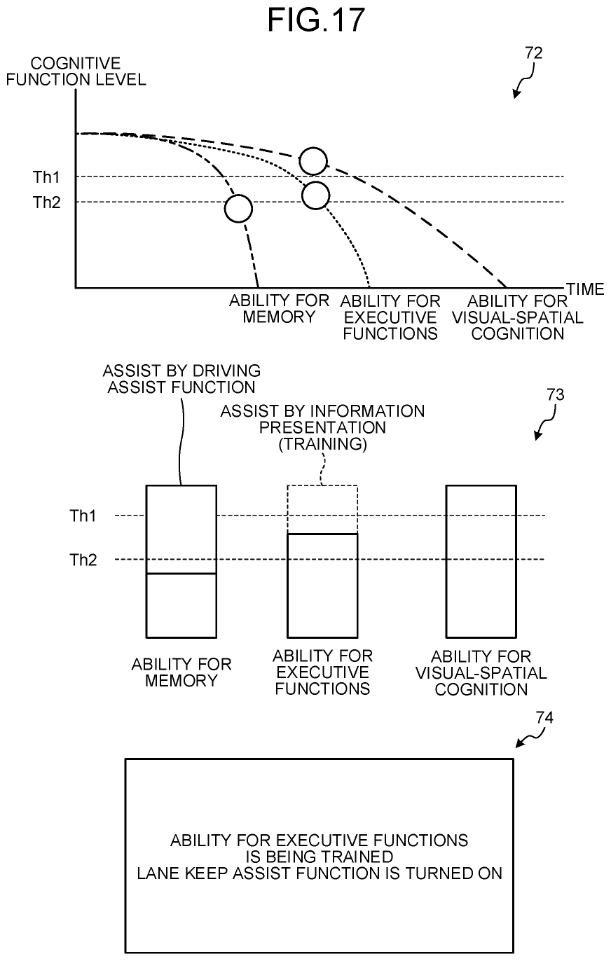

Next, with reference to FIG. 13 to FIG. 17, the following describes an example of information presented to the driver by the driving characteristic determination device FIG. 13 and FIG. 14 are diagrams illustrating an example of the information presented to the vehicle in a case where the driving characteristic determination device activates the training mode. FIG. 15 and FIG. 16 are diagrams illustrating an example of the information presented to the vehicle in a case where the driving characteristic determination device activates the driving assist mode. FIG. 17 is a diagram illustrating an example of the information presented to the vehicle in a case where the driving characteristic determination device activates the training mode and the driving assist mode at the same time.

The cognitive function characteristic output unit 46 outputs information of an analysis result obtained by the cognitive function characteristic analysis unit 44 to the center monitor 25a of the vehicle 30. A presentation screen 64 and a presentation screen 66 illustrated in FIG. 13 are examples of a screen displayed on the center monitor 25a.

The presentation screen 64 is an example of displaying the analysis result obtained by the cognitive function characteristic analysis unit 44 as a radar chart 65. On the radar chart 65, for example, an analysis result from one month ago and an analysis result at the present time are overlapped to be displayed. The driver is able to understand a state of the cognitive functions of himself/herself by checking the presentation screen 64. At this point, a voice message such as “Your ability for attention is declining. Pay attention to surroundings.” may be output from a speaker of the vehicle 30.

The presentation screen 66 is another display example of the analysis result obtained by the cognitive function characteristic analysis unit 44. On the left side of the presentation screen 66, time-series progression 67 of analysis results obtained by the cognitive function characteristic analysis unit 44 is displayed. On the right side of the presentation screen 66, a current analysis result 68 is displayed while being enlarged. In the analysis result 68, the cognitive function at an attention level or a dangerous level may be highlighted in yellow or red. The driver is able to understand a state of the cognitive functions of himself/herself by checking the presentation screen 66.

The assist content display unit 48 displays the assist content determined by the assist content determination unit 47 on the center monitor 25a of the vehicle 30. A presentation screen 69 illustrated in FIG. 14 is an example thereof. On the left side of the presentation screen 69, the analysis result obtained by the cognitive function characteristic analysis unit 44 is displayed for each of the cognitive functions. To a space of the ability for attention 82 for which the assist content determination unit 47 determines to activate the training mode, character information indicating that training is being performed is added. On the right side of the presentation screen 69, an icon indicating that the ability for attention 82 is being trained is displayed. By checking the presentation screen 69, the driver is able to understand that the training mode for the ability for attention 82 is being activated, as well as understanding the state of the cognitive functions of himself/herself.

A presentation screen 70 illustrated in FIG. 15 is an example of a screen indicating that the driving assist mode is being activated, which is displayed on the center monitor 25a of the vehicle 30 by the assist content display unit 48. The presentation screen 70 represents that an inter-vehicle distance following function and a pedestrian detection function are being activated (in an ON state) out of driving assist functions of the vehicle 30, and the other functions are not activated (in an OFF state). The driver is able to understand an operation state of the driving assist functions by checking the presentation screen 70.

A presentation screen 71 illustrated in FIG. 16 is another example of a screen that is displayed on the center monitor 25a of the vehicle 30 by the assist content display unit 48. On the left side of the presentation screen 71, the analysis result obtained by the cognitive function characteristic analysis unit 44 is displayed for each of the cognitive functions. On the right side of the presentation screen 71, information indicating activated driving assist functions out of the driving assist functions of the vehicle 30 is displayed. The presentation screen 71 represents that a detailed guidance function, a pedestrian detection function, and a collision warning are being activated as the driving assist functions for assisting the ability for attention 82 because the ability for attention 82 is at the dangerous level. The driver is able to understand the state of the cognitive functions of himself/herself and the operating state of the driving assist functions by checking the presentation screen 71.

A presentation screen 74 illustrated in FIG. 17 is an example of a screen that is displayed on the center monitor 25a of the vehicle 30 by the assist content display unit 48 in a case where the driving characteristic determination device 10 activates the training mode and the driving assist mode at the same time.

A cognitive function characteristic 72 illustrated in FIG. 17 indicates an example of temporal changes of the ability for memory, the ability for executive function, and the ability for visual-spatial cognition out of the cognitive function characteristics of a driver. Each of circles indicated in FIG. 17 represents the evaluation score of each of the cognitive functions at a specific time. In this case, the ability for memory is smaller than the second threshold Th2. The ability for executive function is between the first threshold Th1 and the second threshold Th2. The ability for visual-spatial cognition is larger than the first threshold Th1.

At this point, as shown in an assist content 73 illustrated in FIG. 17, the assist content determination unit 47 determines that the driving assist mode related to the ability for attention and the training mode related to the ability for executive function should be activated.

The assist content display unit 48 then displays the presentation screen 74 on the center monitor 25a of the vehicle 30. The presentation screen 74 includes character information indicating that the training mode for the ability for executive function is being activated and a lane keep assist installed in the vehicle 30 is being activated. The operating state of the driving assist function is more important than the operating state of the training mode. Therefore, it is preferable that the message indicating that the lane keep assist is being activated is displayed in red or the like, which can attract more attention of the driver, on the presentation screen 74. The operating state of the driving assist function may be displayed in bold. The driver can grasp the operating state of assist functions of the vehicle 30 by checking the presentation screen 74.

The examples of the information that is displayed on the center monitor 25a of the vehicle 30 by the cognitive function characteristic output unit 46 and the assist content display unit 48 have been described above, whereas the driving characteristic determination device 10 may display any piece of the information. Note that, however, it is preferable that a display form is always consistent so as not to confuse the driver. Alternatively, a customizing function for causing the driver to select a display form of information in advance may be provided.

### Operation Example of Training Mode

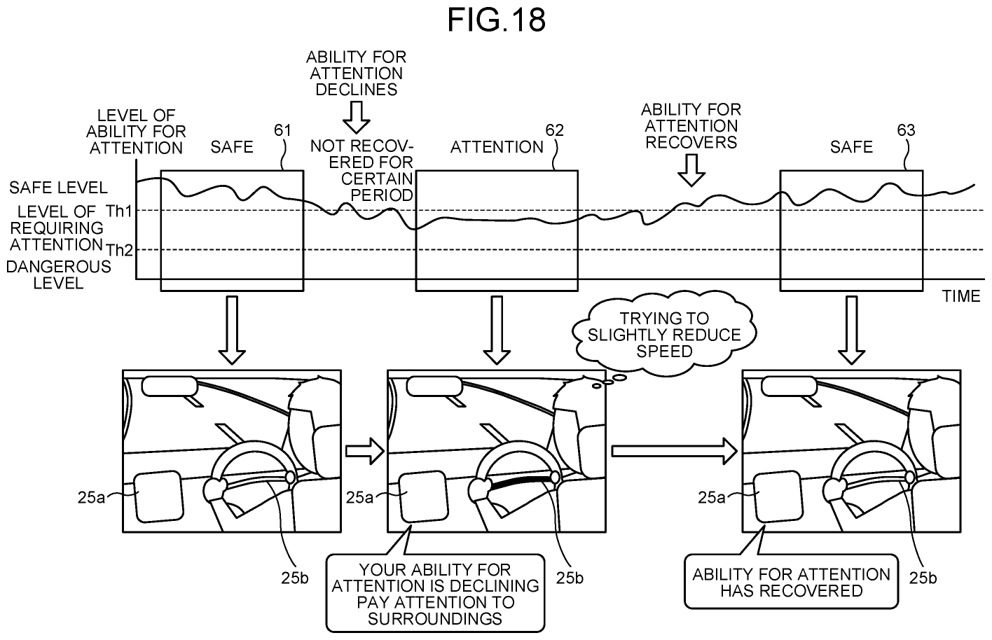

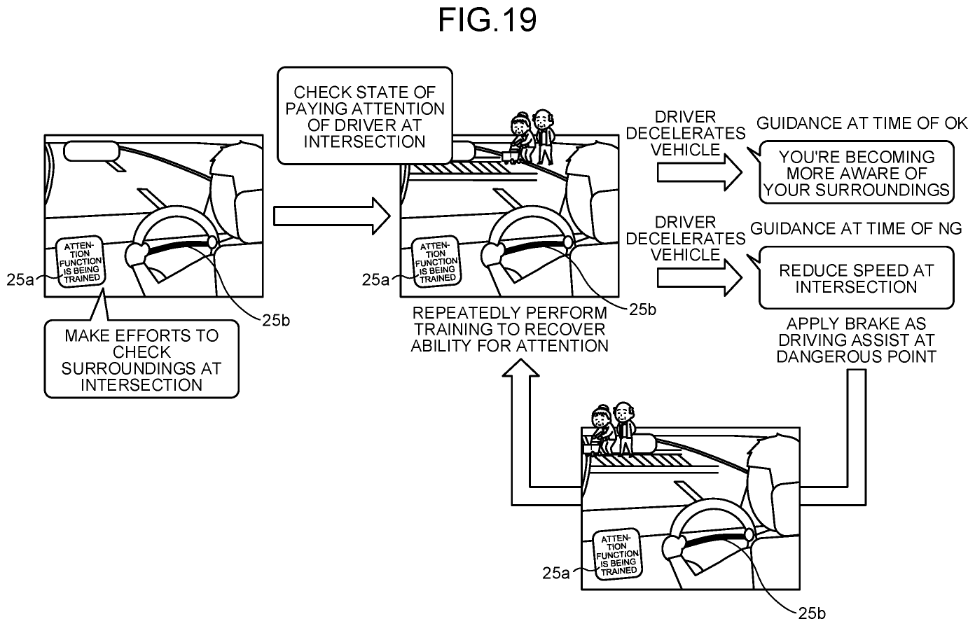

Next, with reference to FIG. 18 and FIG. 19, the following describes an operation example of the training mode. FIG. 18 is a first diagram illustrating an example of an operating state of the training mode. FIG. 19 is a second diagram illustrating an example of an operating state of the training mode.

FIG. 18 illustrates a state where the driving characteristic determination device 10 determines that the ability for attention of the driver has declined and then activates the training mode related to the ability for attention. Specifically, the ability for attention of the driver in a time range 61 is determined to be at a safe level. However, the ability for attention in a time range 62 is determined to be at the level of requiring attention. Therefore, the driving characteristic determination device 10 activates the training mode related to the ability for attention. The ability for attention is recovered to the safe level in a time range 63, so that the driving characteristic determination device 10 ends the training mode related to the ability for attention.

Note that, in a case of activating the training mode or the driving assist mode, it is preferable to perform determination on the basis of an average value or the like of the evaluation scores of the cognitive function in a time range (for example, 15 minutes) such as illustrated in FIG. 18, instead of performing determination on the basis of only an evaluation score of the cognitive function at a specific time point.

When the training mode functions, the assist information presentation unit 49 presents, to the center monitor 25a of the vehicle 30, information for preventing a driving mistake caused by decline of the ability for attention of the driver corresponding to the driving environment of the vehicle 30 detected by the driving environment detection unit 40. For example, information for assisting driving such as “Your ability for attention is declining. Pay attention to surroundings.” is presented. The driver is motivated to reduce the vehicle speed, for example, by checking this display.

The assist content display unit 48 causes the indicator 25b of the vehicle 30 to light in color corresponding to the training mode. The indicator 25b is lit in color corresponding to the driving assist mode when the driving assist mode is being activated, and lit in color corresponding to a state where both of the training mode and the driving assist mode are being activated when both of the training mode and the driving assist mode are being activated.

Moreover, the assist content display unit 48 displays, on the center monitor 25a, a state of the cognitive function of the driver output by the cognitive function characteristic output unit 46 (for example, the presentation screens 64 and 66 in FIG. 13).

FIG. 19 illustrates a state where the cognitive function of the driver is recovered when the training mode is performed.

Assume that the driving characteristic determination device 10 activates the training mode for the ability for attention when the ability for attention of the driver is determined to be at the level of requiring attention. At this point, when the vehicle 30 enters the intersection, the assist information presentation unit 49 performs information assist such as “Please keep in mind to check surroundings at intersection” on the center monitor 25a. When the driving environment detection unit 40 detects that the vehicle approaches the intersection, the driving state detection unit 42 detects a gaze direction and orientation of the face of the driver, and determines whether the driver has performed right and left checking. The driving state detection unit 42 also determines whether the vehicle 30 is decelerated before the intersection by detecting the behavior of the vehicle 30.

In a case where determination is made such that the driver has decelerated the vehicle 30 at the intersection and has performed right and left checking, the assist information presentation unit 49 presents a message such as “You're becoming more aware of your surroundings.” on the center monitor 25a.

On the other hand, in a case where determination is made such that the driver has not decelerated the vehicle 30 at the intersection and has not performed right and left checking, the assist information presentation unit 49 presents a message corresponding to the detected behavior of the driver such as “Reduce speed at intersection”, “Pay attention to right and left side at intersection”, etc., on the center monitor 25a.

The driving assist device of the vehicle 30 does not intervene in the training mode. However, in a dangerous case such as a case where the vehicle 30 is not decelerated even when a pedestrian is present at the intersection, the driving assist device of the vehicle 30 may intervene to activate an automatic brake, for example.

The driving characteristic determination device 10 assists recovery of the ability for attention of the driver by repeatedly performing such training described above.

Processing Procedure Performed by Driving Characteristic Determination Device

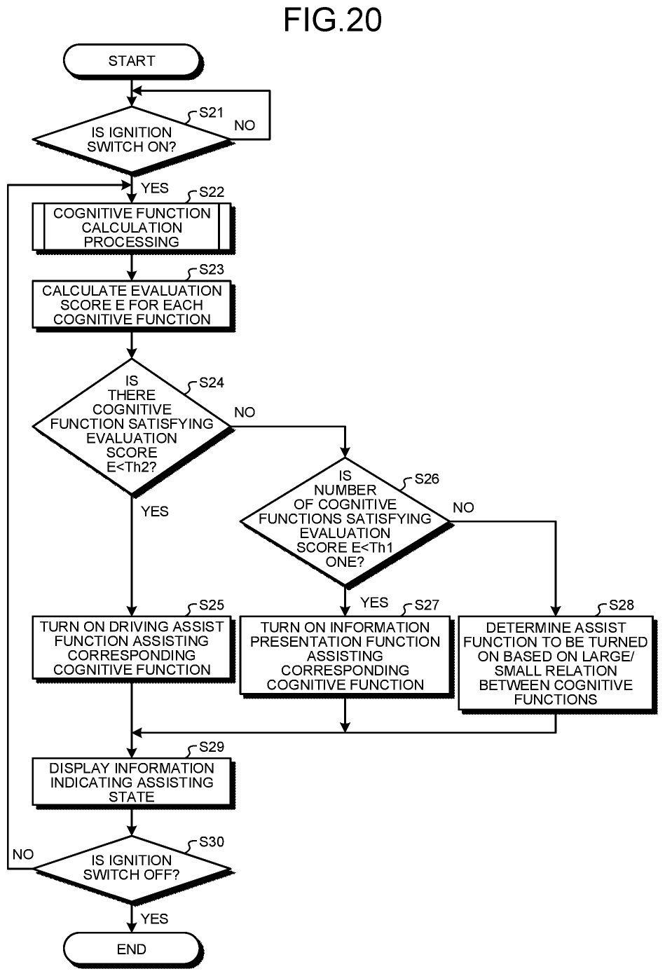

Next, with reference to FIG. 20, the following describes a processing procedure performed by the driving characteristic determination device 10. FIG. 20 is a flowchart illustrating an example of the processing procedure performed by the driving characteristic determination device.

The driving state detection unit 42 determines whether an ignition switch of the vehicle 30 is in an ON state (Step S21). In response to determining that the ignition switch is in the ON state (Yes at Step S21), the process proceeds to Step S22. On the other hand, if it is not determined that the ignition switch is in the ON state (No at Step S21), determination at Step S21 is repeatedly performed.

In response to determining that the ignition switch is in the ON state at Step S21, the driving environment detection unit 40, the driving state detection unit 42, and the cognitive function calculation unit 43 perform cognitive function calculation processing in cooperation with each other (Step S22). The cognitive function calculation processing is performed in accordance with the flowchart described above with reference to FIG. 7.

Subsequently, the cognitive function characteristic analysis unit 44 calculates the evaluation scores Ea, Eb, Ec, Ed, and Ee of the respective cognitive functions relative to different brain functions, on the basis of the cognitive functions obtained through the cognitive function calculation processing (Step S23).

The assist content determination unit 47 determines whether there is a cognitive function whose evaluation score is smaller than the second threshold Th2 (Step S24). In response to determining that there is the cognitive function whose evaluation score is smaller than the second threshold Th2 (Yes at Step S24), the process proceeds to Step S25. On the other hand, when there is not the cognitive function whose evaluation score is smaller than the second threshold Th2 (No at Step S24), the process proceeds to Step S26.

In response to determining that there is the cognitive function whose evaluation score is smaller than the second threshold Th2 at Step S24, the assist content determination unit 47 activates the driving assist function for assisting the corresponding cognitive function (Step S25). Thereafter, the process proceeds to Step S29.

When there is not the cognitive function whose evaluation score is smaller than the second threshold Th2 at Step S24, the assist content determination unit 47 determines whether the number of cognitive functions whose evaluation score is smaller than the first threshold Th1 is one (Step S26). In response to determining that the number of cognitive functions whose evaluation score is smaller than the first threshold Th1 is one (Yes at Step S26), the process proceeds to Step S27. On the other hand, the number of cognitive functions whose evaluation score is smaller than the first threshold Th1 is not one (No at Step S26), the process proceeds to Step S28.

In response to determining that the number of cognitive functions whose evaluation score is smaller than the first threshold Th1 is one at Step S26, the assist content determination unit 47 activates an information presentation function for assisting the corresponding cognitive function (Step S27). Thereafter, the process proceeds to Step S29.

When the number of cognitive functions whose evaluation score is smaller than the first threshold Th1 is not one at Step S26, the assist content determination unit 47 activates the information presentation function for assisting any one of the cognitive functions and the driving assist function for assisting the other cognitive functions, on the basis of a large/small relation between the evaluation scores of the cognitive functions (Step S28). Thereafter, the process proceeds to Step S29.

Subsequent to Steps S25, S27, and S28, the assist content display unit 48 and the assist information presentation unit 49 display information indicating an assisting state on the center monitor 25a and the indicator 25b of the vehicle 30 (Step S29).

The driving state detection unit 42 determines whether the ignition switch of the vehicle 30 is in an OFF state (Step S30). In response to determining that the ignition switch is in the OFF state (Yes at Step S30), the driving characteristic determination device 10 ends the processing in FIG. 20. On the other hand, when the ignition switch is not in the OFF state (No at Step S30), the process returns to Step S22, and the processing described above is repeated.

Working Effects of Embodiment

As described above, the driving characteristic determination device 10 according to the present embodiment includes the driving state detection unit 42, the cognitive function calculation unit 43, the cognitive function characteristic analysis unit 44, and the cognitive function characteristic output unit 46. The driving state detection unit 42 detects at least one of the driving behavior for the vehicle 30 by the driver, the biological information of the driver during driving, and the behavior of the vehicle 30. The cognitive function calculation unit 43 calculates the evaluation score E (numerical value) indicating whether the cognitive function of the driver is high or low, on the basis of the information detected by the driving state detection unit 42. The cognitive function characteristic analysis unit 44 analyzes the evaluation score E indicating whether the cognitive function is high or low calculated by the cognitive function calculation unit 43. The evaluation score E is analyzed as cognitive function characteristics relative to one or more different brain functions. The cognitive function characteristic output unit 46 outputs the information of the analysis result obtained by the cognitive function characteristic analysis unit 44. Accordingly, the driving behavior of the driver can be appropriately assisted in accordance with the cognitive function characteristics relative to one or more different brain functions of the driver. The driving characteristic determination device 10 can detect a state where the cognitive function has temporarily declined when a healthy driver performs cognitive distracted driving, visual-manual distracted driving, etc., and can also detect a state where the cognitive function has declined due to aging or a state called MCI.

In the driving characteristic determination device 10 according to the present embodiment, the cognitive function characteristic analysis unit 44 calculates the cognitive function characteristic from the information detected by the driving state detection unit 42 on the basis of a correspondence relation between the information detected by the driving state detection unit 42 and a numerical value indicating whether the cognitive function is high or low that is set in advance. Accordingly, the state of the cognitive function of the driver can be easily calculated.

In the driving characteristic determination device 10 according to the present embodiment, the cognitive function characteristic analysis unit 44 detects, on the basis of the driving environment of the vehicle 30, at least one of the driving behavior for the vehicle 30 by the driver, the biological information of the driver during driving, and the behavior of the vehicle 30 that are expected to be caused in the driving environment. Accordingly, a cognitive characteristic is analyzed by using only the driving state that is estimated from the driving environment, out of pieces of the information detected by the driving state detection unit 42. Therefore, a calculation load can be reduced.

The driving characteristic determination device 10 according to the present embodiment further includes the assist content determination unit 47 (determination unit) that determines whether to enable the function of assisting information presentation for suppressing further decline of the cognitive function of the driver, or enable the function of assisting a driving operation relative to the cognitive function characteristic, out of the functions of the vehicle 30 on the basis of comparison between the cognitive function characteristic calculated by the cognitive function characteristic analysis unit 44, and the first threshold Th1 and the second threshold Th2 (thresholds). Accordingly, it is possible to easily determine content of driving assist that is activated.

In the driving characteristic determination device 10 according to the present embodiment, the assist content determination unit 47 (determination unit) determines, for the cognitive function smaller than the threshold, whether to enable the function of assisting information presentation for suppressing further decline of the cognitive function, or enable the function of assisting a driving operation relative to the cognitive function. Accordingly, a driving assist corresponding to the cognitive function of the driver can be performed.

In the driving characteristic determination device 10 according to the present embodiment, the assist content determination unit 47 (determination unit) enables the function of assisting the driving operation relative to the cognitive function characteristic in a case where the cognitive function is smaller than the second threshold Th2 that is smaller than the first threshold Th1, and enables the function of assisting information presentation for suppressing further decline of the cognitive function in a case where the cognitive function is smaller than the first threshold Th1 and larger than the second threshold Th2. Accordingly, a driving assist corresponding to the cognitive function of the driver can be performed. For example, for the driver having the cognitive function at the level of requiring attention, recovery of the cognitive function can be promoted by activating the training mode by information presentation. On the other hand, for the driver having the cognitive function at the dangerous level, the declined cognitive function can be executed by the vehicle 30 by activating the driving assist function.

In the driving characteristic determination device 10 according to the present embodiment, in a case where the cognitive functions relative to different brain functions are smaller than the first threshold Th1 and larger than the second threshold Th2, the assist content determination unit 47 (determination unit) determines whether to enable the function of assisting information presentation for suppressing further decline of the cognitive function, or enable the function of assisting the driving operation relative to the cognitive function for each of the cognitive functions. Accordingly, in a case where the cognitive functions have declined to the same degree, it is possible to determine the cognitive function to be assisted by information presentation and the cognitive function to be assisted by driving assist.

In the driving characteristic determination device 10 according to the present embodiment, the cognitive function characteristic output unit 46 (output unit) further outputs the state of the cognitive function characteristics relative to one or more different brain functions calculated by the cognitive function characteristic analysis unit 44. Accordingly, it is possible to visualize and present, to the driver, the state of the cognitive functions of himself/herself.

The driving characteristic determination device 10 according to the present embodiment further includes the driver specification unit 41 (specification unit) that specifies the driver. Accordingly, the cognitive characteristic of the same driver can be continuously analyzed.

First Modification of Embodiment

As a first modification of the embodiment described above, the following describes an example in which the driving characteristic determination device 10 analyzes temporal changes of the cognitive function characteristic of the same driver.

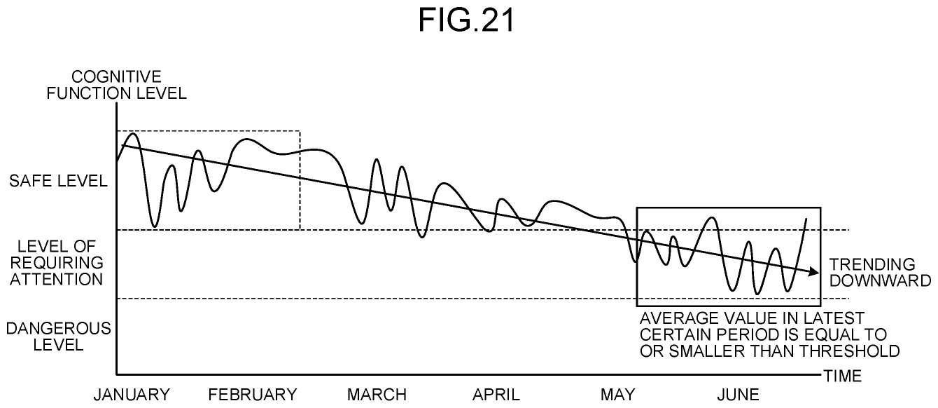

FIG. 21 is a diagram for explaining an effect of the modification of the embodiment. The driver specification unit 41 (refer to FIG. 5) included in the driving characteristic determination device 10 specifies the driver who is driving the vehicle 30. The driving characteristic determination device 10 stores, in the cognitive function storage unit 45, the evaluation score E of the cognitive function acquired in the past in association with the driver. Accordingly, when the driver has been specified, the driving characteristic determination device 10 can read the evaluation score E in the past, which is associated with the specified driver.

The temporal changes of the cognitive function illustrated in FIG. 21 represent progression of the evaluation score E of the cognitive function of the driver specified by the driver specification unit 41. A vertical axis in FIG. 21 can also indicate the cognitive function characteristics relative to different brain functions (the ability for memory, the ability for executive function, the ability for attention, the ability for information processing, and the ability for visual-spatial cognition).

The cognitive function characteristic analysis unit 44 analyzes information of the temporal changes of the cognitive function illustrated in FIG. 21. For example, in a case where an average value of the evaluation score E in the latest certain period is determined to be at the level of requiring attention, the cognitive function characteristic notification unit 51 (refer to FIG. 5) makes notification of data indicating the temporal changes of the cognitive function, to a transmission destination registered in advance such as a family of the driver. At this point, a message such as “Cognitive function required for safe driving tends to be declining. Taking driving lessons is recommended.” may be added thereto.

Conversely, the family of the driver may request the cognitive function notification unit to transmit the data of temporal changes of the cognitive function of the driver.

As described above, the driving characteristic determination device 10 according to the first modification of the present embodiment further includes the cognitive function characteristic notification unit 51 (notification unit) that makes notification about temporal changes of the cognitive function of the same driver. Accordingly, temporal changes of the cognitive function of the driver can be monitored over a long period. Accordingly, there is the possibility that a state of MCI in which the cognitive function has declined due to aging and dementia appears can be detected at an early stage.

Second Modification of Embodiment

In the embodiment described above, by using at least one of the driving behavior of the driver, the biological information of the driver during driving, and the behavior of the vehicle 30 detected by the driving state detection unit 42, the cognitive function calculation unit 43 and the cognitive function characteristic analysis unit 44 calculate the cognitive function of the driver using a table indicating a relation between the detection result of the driving state and the evaluation score that is created in advance. On the other hand, in a second modification described below, the cognitive function characteristic of the driver is analyzed by using a driving behavior model that has been learned in advance.

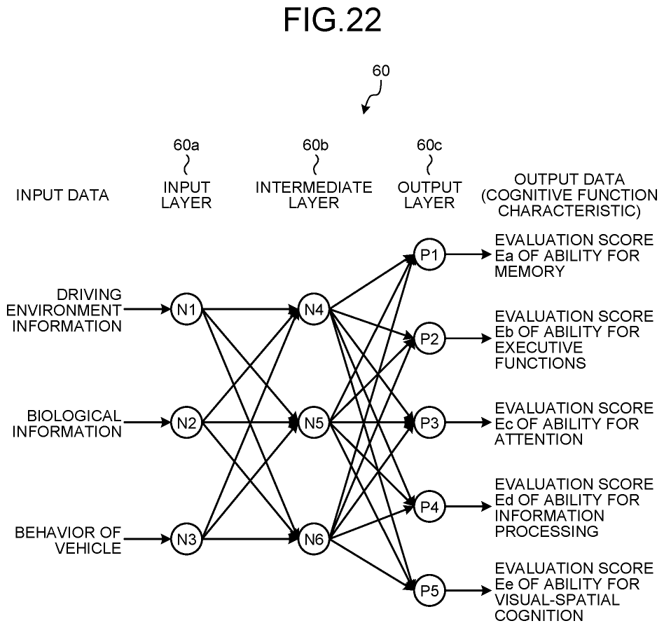

FIG. 22 is a diagram for explaining another method for calculating the cognitive function characteristic. A driving behavior model 60 illustrated in FIG. 22 receives, as inputs, driving environment information of the vehicle 30 detected by the driving environment detection unit 40, and the biological information of the driver and the behavior of the vehicle 30 detected by the driving state detection unit 42, and outputs the evaluation score Ea of the ability for memory 80, the evaluation score Eb of the ability for executive function 81, the evaluation score Ec of the ability for attention 82, the evaluation score Ed of the ability for information processing 83, and the evaluation score Ed of the ability for visual-spatial cognition 84. The information to be input does not include the information related to the driving behavior of the driver described above in the embodiment. Typically, the information related to the driving behavior of the driver can be calculated on the basis of the driving environment information of the vehicle 30 and the biological information of the driver. Therefore, it is automatically calculated inside the driving behavior model 60.

Various methods can be considered as a method for writing the driving behavior model 60, whereas the present modification uses a model that has been learned by means of deep learning. The driving behavior model 60 illustrated in FIG. 22 is constituted of a neural network including an input layer 60a, an intermediate layer 60b, and an output layer 60c. The neural network is a mathematical model imitating a nerve network of a person.

The input layer 60a includes three input units N1, N2, and N3. Values corresponding to the driving environment information, the biological information, and the behavior of the vehicle 30 are input to the input units N1, N2, and N3, respectively.

The value input to the input layer 60a is output to the intermediate layer 60b. At this point, the value input from the input layer 60a is integrated with weighting factors given to nodes connecting the input units N1, N2, and N3 with intermediate units N4, N5, and N6 of the intermediate layer 60b. Integrated numerical values are added up in the respective intermediate units N4, N5, and N6.

The output layer 60c includes five output units P1, P2, P3, P4, and P5. Each of the output units P1, P2, P3, P4, and P5 is connected to the intermediate units N4, N5, and N6 via nodes to which weighting factors are given.

Values output from the intermediate units N4, N5, and N6 are integrated with the weighting factors given to nodes connecting the intermediate units with the output units. Integrated numerical values are added up in the respective output units P1, P2, P3, P4, and P5.

The output units P1, P2, P3, P4, and P5 respectively output the added values. The weighting factors of respective nodes included in the driving behavior model 60 are tuned by performing learning such that the output values become values corresponding to the evaluation scores Ea, Eb, Ec, Ed, and Ee of the respective cognitive functions.

By using the driving behavior model 60 that is formed as described above, the evaluation scores Ea, Eb, Ec, Ed, and Ee of the cognitive functions relative to one or more different brain functions can be obtained on the basis of the driving environment information of the vehicle 30 detected by the driving environment detection unit 40 and the biological information of the driver and the behavior of the vehicle 30 detected by the driving state detection unit 42.

Note that a form of the driving behavior model 60 is not limited to the example illustrated in FIG. 22. For example, the intermediate layer 60b may be constituted of a plurality of layers. Additionally, the number of the intermediate units is not limited to three.

As described above, in the driving characteristic determination device 10 according to the second modification of the present embodiment, the cognitive function characteristic analysis unit 44 analyzes, as the cognitive function characteristics relative to one or more different brain functions, the numerical values indicating whether the cognitive function calculated by the cognitive function calculation unit 43 is high or low on the basis of the information detected by the driving state detection unit 42 and the driving behavior model 60 that has been learned in advance. Accordingly, the evaluation scores Ea, Eb, Ec, Ed, and Ee of the cognitive functions relative to one or more different brain functions can be easily obtained without performing a complicated arithmetic operation or referring to a table.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

With the driving characteristic determination device according to the present disclosure, a driving mistake that tends to be made by a driver can be estimated by analyzing a cognitive function characteristic of the driver on the basis of a cognitive mechanism of a brain. By using a result thereof, it is possible to assist driving behavior of the driver, or train the cognitive function.

What is claimed is:

1. A driving characteristic determination device comprising
a hardware processor connected to a memory, the hardware processor being configured to:
detect driving information indicating at least one of driving behavior for a vehicle by a driver, biological information of the driver during driving, and behavior of the vehicle;
calculate, on the basis of the driving information, numerical values indicating whether a cognitive function of the driver is high or low;
analyze the numerical values indicating whether the cognitive function is high or low, the numerical values being analyzed as cognitive function characteristics relative to one or more different brain functions; and
output information about an analysis result obtained by the analysis of the numerical values.

2. The driving characteristic determination device according to claim 1, wherein the hardware processor is configured to calculate the cognitive function characteristic from the driving information on the basis of a correspondence relation between the driving information and the numerical values indicating whether the cognitive function is high or low, the correspondence relation being set in advance.

3. The driving characteristic determination device according to claim 1, wherein the hardware processor is configured to detect the driving information on the basis of driving environment of the vehicle, the driving information indicating at least one of the driving behavior for the vehicle by the driver, the biological information of the driver during driving, and the behavior of the vehicle that are expected to be caused in the driving environment.

4. The driving characteristic determination device according to claim 1, wherein the hardware processor is further configured to determine, from among a plurality of functions of the vehicle, whether to enable a function of assisting information presentation for suppressing further decline of the cognitive function of the driver, or enable a function of assisting a driving operation relative to the cognitive function characteristic, the determination being performed on the basis of comparison between a threshold and the cognitive function characteristic.

5. The driving characteristic determination device according to claim 4, wherein the hardware processor is configured to determine, for a cognitive function smaller than the threshold, whether to enable the function of assisting information presentation for suppressing further decline of the cognitive function, or enable the function of assisting a driving operation relative to the cognitive function.

6. The driving characteristic determination device according to claim 4, wherein the hardware processor is configured to
enable the function of assisting a driving operation relative to the cognitive function characteristic in a case where the cognitive function is smaller than a second threshold, the second threshold being smaller than a first threshold, and
enable the function of assisting information presentation for suppressing further decline of the cognitive function in a case where the cognitive function is smaller than the first threshold and larger than the second threshold.

7. The driving characteristic determination device according to claim 6, wherein, in a case where cognitive functions each being relative to a different one of brain functions are smaller than the first threshold and larger than the second threshold, the hardware processor is configured to determine, for each of the cognitive functions, whether to enable the function of assisting information presentation for suppressing further decline of the cognitive function, or enable the function of assisting a driving operation relative to the cognitive function.

8. The driving characteristic determination device according to claim 1, wherein the hardware processor is configured to further output information representing states of cognitive function characteristics having been analyzed, the cognitive function characteristics being relative to one or more different brain functions.

9. The driving characteristic determination device according to claim 1, wherein the hardware processor is configured to perform the analysis of the numerical values on the basis of the driving information and a driving behavior model having been learned in advance.

10. The driving characteristic determination device according to claim 1, wherein the hardware processor is further configured to specify the driver.

11. The driving characteristic determination device according to claim 1, wherein the hardware processor is further configured to makes notification about temporal changes of the cognitive function of the same driver.

12. A driving characteristic determination method comprising:
detecting driving information indicating at least one of driving behavior for a vehicle by a driver, biological information of the driver during driving, and behavior of the vehicle;
calculating, on the basis of the driving information, numerical values indicating whether a cognitive function of the driver is high or low;
analyzing the numerical values indicating whether the cognitive function is high or low, the numerical values being analyzed as cognitive function characteristics relative to one or more different brain functions; and
outputting information about an analysis result obtained by the analyzing of the numerical values.

13. The driving characteristic determination method according to claim 12, wherein the analyzing of the numerical values is performed on the basis of the driving information and a driving behavior model having been learned in advance.

14. A non-transitory computer-readable recording medium on which programmed instructions are recorded, the instructions causing a computer to execute processing including:
detecting driving information indicating at least one of driving behavior for a vehicle by a driver, biological information of the driver during driving, and behavior of the vehicle;
calculating, on the basis of the driving information, numerical values indicating whether a cognitive function of the driver is high or low;
analyzing the numerical values indicating whether the cognitive function is high or low, the numerical values being analyzed as cognitive function characteristics relative to one or more different brain functions; and
outputting information about an analysis result obtained by the analyzing of the numerical values.

15. The non-transitory computer-readable recording medium according to claim 14, wherein the analyzing of the numerical values is performed on the basis of the driving information and a driving behavior model having been learned in advance.
